(19) United States
(12) Patent Application Publication
(10) Pub. No.: US 2019/0175519 A1
(43) Pub. Date: Jun. 13, 2019

(54) Naloxone Transdermal Delivery Devices and Methods for Using the Same

(71) Applicant: Teikoku Pharma USA, Inc., San Jose, CA (US)

(72) Inventors: Russell Haynes, San Jose, CA (US); Jianye Wen, Palo Alto, CA (US); Jutaro Shudo, San Jose, CA (US); Bret Berner, Half Moon Bay, CA (US)

(21) Appl. No.: 16/213,574

(22) Filed: Dec. 7, 2018

Related U.S. Application Data
(60) Provisional application No. 62/596,660, filed on Dec. 8, 2017.

(51) Int. Cl. A61K 9/70; A61K 31/485; A61K 47/12

(52) U.S. Cl. CPC A61K 9/7053; A61K 47/12; A61K 9/7061; A61K 31/485

(57) ABSTRACT

Naloxone transdermal delivery devices are provided. Aspects of the naloxone transdermal delivery devices include a matrix comprising a naloxone active agent in a pressure sensitive adhesive and a backing layer. Also provided are methods of making and using the naloxone transdermal delivery devices, as well as kits containing the transdermal delivery devices. The devices, kits and methods of using the same find use in a variety of applications.

### CROSS REFERENCE TO RELATED APPLICATION

Pursuant to 35 U.S.C. § 119(e), this application claims priority to the filing date of U.S. Provisional Patent Application Ser. No. 62/596,660, filed Dec. 8, 2017; the disclosure of which application is herein incorporated by reference.

### INTRODUCTION

Naloxone is a narcotic antagonist that prevents or reverses the effects of opioids. Naloxone (i.e., 17-allyl-4,5α-epoxy-3,14-dihydroxymorphinan-6-one), also known as Narcan and Evzio, is described by the formula:

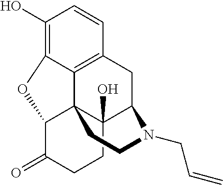

The compound and methods for its synthesis are described in U.S. Pat. No. 3,254,088. Its use as a narcotic antagonist is described in U.S. Pat. No. 4,267,182.

A preferred route of administration of a narcotic antagonist is by the parenteral route (i.e. intravenous injection or infusion). The parenteral route of administration affords rapid delivery of the drug, complete bioavailability, and is more predictable and controllable than other routes. Solution formulations for parenteral administration must be essentially free of particulate matter, and they must be sterile. They must be physically and chemically stable, so that efficacy and safety are predictable.

### SUMMARY

Naloxone transdermal delivery devices are provided. Aspects of the naloxone transdermal delivery devices include a matrix comprising a naloxone active agent in a pressure sensitive adhesive and a backing layer. Also provided are methods of making and using the naloxone transdermal delivery devices, as well as kits containing the transdermal delivery devices. The devices, kits and methods of using the same find use in a variety of applications.

### BRIEF DESCRIPTION OF THE FIGURES

FIG. 1 depicts a comparison of transdermal flux for naloxone transdermal delivery devices having different concentrations of naloxone and different permeation enhancers along with a clonidine transdermal delivery device as a control.

### DETAILED DESCRIPTION

Naloxone transdermal delivery devices are provided. Aspects of the naloxone transdermal delivery devices include a matrix comprising a naloxone active agent in a pressure sensitive adhesive and a backing layer. Also provided are methods of making and using the naloxone transdermal delivery devices, as well as kits containing the transdermal delivery devices. The devices, kits and methods of using the same find use in a variety of applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various embodiments of the invention, aspects of the naloxone transdermal delivery devices are reviewed first in greater detail, followed by a description of embodiments of using the transdermal delivery devices and a review of kits that include the subject extended transdermal delivery devices.

### Naloxone Transdermal Delivery Devices

As summarized above, aspects of the invention include naloxone transdermal delivery devices. In embodiments, the transdermal delivery devices include: (a) a matrix that includes a naloxone active agent present in a pressure sensitive adhesive; and (b) a backing layer.

Naloxone (i.e., 17-allyl-4,5α-epoxy-3,14-dihydroxymorphinan-6-one) is described by the formula:

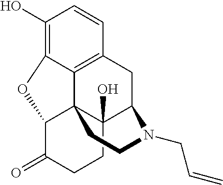

A naloxone active agent according to embodiments of the invention may be in the form of a free base, salt, solvate, hydrate, co-crystal or complex. For example, naloxone may be in the form of a pharmaceutically acceptable salt including, but not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2] oct-2-ene 1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. In certain embodiments, the naloxone active agent is naloxone HCl. In other embodiments, the naloxone active agent is naloxone free base. In yet other instances, the naloxone active agent forms a complex.

In some instances, the naloxone transdermal delivery devices are configured to provide for one or more of the following naloxone delivery features. Transdermal administration of naloxone is, in some embodiments, passive. By “passive” transport is meant that the naloxone active agent is delivered from the matrix across the skin or mucous membrane in the absence of applied energy (e.g., rubbing or heat) and is primarily dependent on the permeability of the barrier (e.g., skin or mucous membrane) and by the thermodynamic activity or concentration gradient across the stratum corneum.

In some embodiments, the naloxone transdermal delivery devices are configured to provide for extended transdermal delivery of naloxone to a subject. By “extended transdermal delivery” is meant delivery of the naloxone to the subject over an extended period of time, such as over the course of hours, days and including weeks, including 1 hour or longer, such as 2 hours or longer, such as 4 hours or longer, such as 8 hours or longer, such as 12 hours or longer, such as 24 hours (1 day) or longer, such as 48 hours (2 days) or longer, such as 72 hours (3 days) or longer, such as 96 hours (4 days) or longer, such as 120 hours (5 days) or longer, such as 144 hours (6 days) or longer and including 168 hours (7 days) or longer. For the above ranges an upper limit period of time is, in some instances, 168 hours or shorter, such as 144 hours or shorter, such as 120 hours or shorter, such as 96 hours or shorter, such as 72 hours or shorter, such as 48 hours or shorter and including 24 hours or shorter. In certain embodiments, extended transdermal delivery ranges from 0.5 hours to 168 hours, such as from 1 hour to 144 hours, such as from 1.5 hours to 120 hours, such from 2 hours to 96 hours, such as from 2.5 hours to 72 hours, such as from 3 hours to 48 hours, such as from 3.5 hours to 24 hours, such as from 4 hours to 12 hours and including from 5 hours to 8 hours.

In some embodiments, the naloxone transdermal delivery devices are configured to deliver a target dosage of naloxone to the subject, such as for example delivering a target dosage as determined by total drug exposure or by average daily drug exposure. Depending on the desired therapeutic effect of the naloxone transdermal delivery device, the treatment protocol and the physiology of the subject, target drug exposure may vary. In certain embodiments, the target drug exposure of naloxone is an amount which is in the therapeutic window of the subject. In embodiments of the invention, a therapeutically effective amount provides for a systemic amount of naloxone that enables the desired treatment. The dosage delivered by a given naloxone transdermal delivery device may vary, where in some instances the delivered dosage of naloxone ranges from 0.1 μg/day to 10,000 μg/day, such as from 1 μg/day to 8,000 μg/day, including from 50 μg/day to 5,000 μg/day, e.g., from 100 μg/day to 3,000 μg/day.

In some embodiments, the delivered dosage for which the transdermal delivery device is configured to administer is an amount that provides for a systemic amount of naloxone that results in a desired mean plasma concentration of naloxone at specific times during treatment. In other embodiments, the delivered dosage is an amount that when applied to a subject provides for a steady state mean plasma concentration of naloxone throughout a dosage interval or treatment protocol. In yet other embodiments, the delivered dosage is an amount that when applied to a subject provides for a particular rate of delivery of naloxone to the subject.

In some embodiments, the naloxone transdermal delivery devices are configured to deliver an average cumulative systemic amount of naloxone delivered over the course of a dosage interval (e.g., 1 day or longer, such as 3 days or longer). The term “average cumulative systemic amount” is used to refer to the total quantity of naloxone which is delivered to the subject through the skin and is absorbed systemically. In some embodiments, the average cumulative systemic amount of delivered naloxone may be 0.01 μg/cm² or greater, such as 0.1 μg/cm² or greater, e.g., 1 μg/cm² or greater, including 10 μg/cm², and in some instances up to 50 μg/cm² or greater over a dosage interval, such as a 1, 3 or 7 day dosage interval.

In other embodiments, the delivered dosage for which the delivery device is configured to administer is an amount that provides for a local amount of naloxone that results in a desired therapeutic activity. In these embodiments, the naloxone transdermal delivery devices may be configured to deliver an average cumulative local amount of naloxone delivered over the course of a dosage interval (e.g., 1 day or longer, such as 3 days or longer). The term “average cumulative local amount” is used to refer to the total quantity of naloxone which is delivered locally to the subject through the skin. In some embodiments, the average cumulative local amount of delivered naloxone may be 0.01 μg/cm² or greater, such as 0.1 μg/cm² or greater, e.g., 1 μg/cm² or greater, including 10 g/cm², and in some instances up to 50 μg/cm² or greater over a dosage interval, such as a 1, 3 or 7 day dosage interval.

In some instances, the naloxone transdermal delivery devices are configured to provide a peak naloxone flux of 10 μg/cm²/hr or less, such as 9 g/cm²/hr or less, such as 8 μg/cm²/hr or less, such as 7 μg/cm²/hr or less, such as 6 μg/cm²/hr or less, such as 5 μg/cm²/hr or less, such as 4 μg/cm²/hr or less, such as 3 μg/cm²/hr or less, such as 2 μg/cm²/hr or less, such as 1 μg/cm²/hr or less, such as 0.5 μg/cm²/hr or less and including 0.1 μg/cm²/hr or less. In some embodiments, the naloxone transdermal delivery device is configured to provide a peak naloxone flux that ranges from 0.01 μg/cm²/hr to 15 μg/cm²/hr, such as from 0.05 μg/cm²/hr to 14 μg/cm²/hr, such as from 0.1 μg/cm²/hr to 13 μg/cm²/hr, such as from 0.5 μg/cm²/hr to 12 μg/cm²/hr, such as from 1 μg/cm²/hr to 11 g/cm²/hr, such as from 2 μg/cm²/hr to 10 μg/cm²/hr, such as from 3 μg/cm²/hr to 9 μg/cm²/hr and including from 4 μg/cm²/hr to 8 μg/cm²/hr.

In certain embodiments, the naloxone transdermal delivery devices are configured to provide, after an initial post-application period, a steady state average flux of naloxone to a subject. By initial post-application period is meant a period of time following initial application of the device to the skin. In some instances, the initial post-application period is a duration of 1 minute or more, such as 2 minutes or more, such as 3 minutes or more, such as 5 minutes or more, such as 10 minutes or more, such as 15 minutes or more, such as 30 minutes or more, such as 45 minutes or more and including 60 minutes or more. For example, the initial post-application period may be a duration of from 1 minute to 120 minutes, such as from 2 minutes to 90 minutes, such as from 3 minutes to 60 minutes and including from 5 minutes to 30 minutes. In certain embodiments, the naloxone transdermal delivery devices are configured to provide a steady state average flux of naloxone to a subject after 12 hours or more, such as where the initial post-application period is 15 hours or more, such as 18 hours or more, such as 24 hours or more, such as 36 hours or more, such as 48 hours or more and including 72 hours or more. For example, the initial post-application period may be a duration of from 12 hours to 96 hours, such as from 18 hours to 90 hours, such as from 24 hours to 84 hours, such as from 30 hours to 78 hours and including from 36 hours to 72 hours.

Following this initial post-application period, in some instances the naloxone flux from transdermal delivery devices of interest decreases by 80% or less, such as 60% or less, including 30% or less during the time while the transdermal delivery device is maintained in contact with the subject over the application duration for which the device is configured, such as 25% or less, such as 20% or less, such as 15% or less, such as 12% or less, such as 10% or less, such as 6% or less, such as 5% or less, such as 4% or less, and including 1% or less during the time while the transdermal delivery device is maintained in contact with the subject over the application duration for which the device is configured. In some instances, the devices are configured to maintain the steady state average naloxone flux for 0.5 hours or longer, such as 1 hour or longer, such as 2 hours or longer, such as 3 hours or longer, such as 4 hours or longer, such as 8 hours or longer, 12 hours or longer, such as 24 hours or longer, such as 36 hours or longer, such as 48 hours or longer, such as 72 hours or longer, such as 96 hours or longer, such as 120 hours or longer, such as 144 hours or longer and including 168 hours or longer.

As summarized above, the naloxone transdermal delivery devices of the invention include a matrix that includes a naloxone active agent and a backing. In some embodiments of the invention, the matrix of the transdermal delivery device is configured as a single layer matrix. By “single layer” is meant that the transdermal delivery device includes only a single layer of matrix disposed on the surface of a backing of the transdermal delivery device and does not include separate distinct layers for the pressure sensitive adhesive, transdermal naloxone active agent composition, or if present, any skin penetration enhancers.

Likewise, single layer transdermal delivery devices do not further include a separate naloxone active agent reservoir (i.e., active agent reservoir) separate from the pressure sensitive adhesive. As such, single layer transdermal delivery device embodiments of the invention may include in a single matrix an amount of each of the components of the transdermal naloxone compositions necessary for practicing the subject methods, as described in greater detail below. For example, in some embodiments, single layer transdermal delivery devices include a single layer matrix of a naloxone active agent and a pressure sensitive adhesive, where one or more additional components may be present in the matrix, as desired, e.g., solubility enhancers, cross-linked polymers, etc., such as described in greater detail below. The matrix thickness may vary, in some instances ranging from 10 to 260 microns, such as 15 to 250 microns, such as 25 to 225 microns, such as 50 to 200 microns, such as 75 to 175 microns and including 20 to 130 microns such as 35 to 110 microns. The amount of naloxone active agent in the matrix may also vary. In some instances, the amount of naloxone in the matrix ranges from 0.001 mg to 100 mg, such as from 0.003 mg to 100 mg, such as from 0.005 mg to 95 mg, such as from 0.01 mg to 90 mg, such as from 0.05 mg to 85 mg, such as from 0.1 mg to 80 mg such as from 0.1 to 50 mg, such as from 0.2 to 40 mg, including 1 to 20 mg, as well as ranges in between, such as from 0.001 to 10 mg, such as from 0.005 mg to 9 mg, such as from 0.01 to 7.5 mg, such as from 0.1 mg to 5 mg and including from 0.1 mg to 3 mg. In some embodiments, the amount of naloxone active agent in the matrix of the transdermal delivery device ranges from 0.1% to 20% w/w, such as 0.5% to 18% w/w, such as 1% to 15%, such as 2% to 12.5% w/w and including 3% to 10% w/w.

As reviewed above, the matrix of the transdermal delivery devices also includes a pressure sensitive adhesive. Pressure sensitive adhesives may include, but are not limited to, acrylic or acrylate copolymers, poly-isobutene adhesives, poly-isobutylene adhesives, poly-isobutene/polyisobutylene adhesive mixtures, silicone adhesives, e.g., amine-resistant silicone adhesives, styrene block copolymer adhesives, etc., as well as blends of two or more of these types adhesives

Acrylate copolymers of interest include copolymers of various monomers, such as “soft” monomers, “hard” monomers or “functional” monomers. The acrylate copolymers can be composed of a copolymer including bipolymer (i.e., made with two monomers), a terpolymer (i.e., made with three monomers), or a tetrapolymer (i.e., made with four monomers), or copolymers having greater numbers of monomers. The acrylate copolymers may be crosslinked or non-crosslinked. The polymers can be cross-linked by known methods to provide the desired polymers. The monomers from of the acrylate copolymers may include at least two or more exemplary components selected from the group including acrylic acids, alkyl acrylates, methacrylates, copolymerizable secondary monomers or monomers with functional groups. Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference. In some embodiments, the pressure sensitive adhesive is an acrylate-vinyl acetate copolymer. In some embodiments, the pressure sensitive adhesive may include a composition that is, or is substantially the same as, the composition of Duro-Tak® 87-9301, Duro-Tak® 87-200A, Duro-Tak®87-2353, Duro-Tak®87-2100, Duro-Tak®87-2051, Duro-Tak®87-2052, Duro-Tak®87-2194, Duro-Tak®87-2677, Duro-Tak®87-201A, Duro-Tak®87-2979, Duro-Tak®87-2510, Duro-Tak®87-2516, Duro-Tak®87-387, Duro-Tak®87-4287, Duro-Tak®87-2287, and Duro-Tak®87-2074 and combinations thereof. The term “substantially the same” as used herein refers to a composition that is an acrylate-vinyl acetate copolymer in an organic solvent solution.

For example, acrylate copolymers of interest may be formed from monomers of having the formula:

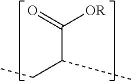

where R is hydrogen, linear alkyl, branched alkyl or a substituted alkyl thereof. In certain embodiments, the pressure sensitive adhesive is an acrylate adhesive that is a non-functionalized acrylate (e.g., where R is alkyl). By non-functionalized is meant that the pressure sensitive adhesive does not include any pendant functional groups, such as are present on hydroxyl-functionalized acrylates or an acid functionalized acrylates. For example, non-functionalized pressure sensitive adhesives are ones that lack pendent hydroxyl (—OH) functional groups. As such, non-functionalized pressure sensitive adhesives do not include one or more —OH functional groups, such as found in the following pressure sensitive adhesives: Duro-Tak® 87-4287, Duro-Tak® 87-2287, Duro-Tak® 87-2510 and Duro-Tak® 87-2516. Likewise, non-functionalized pressure sensitive adhesives are ones that lack pendent acid (e.g., —COOH) functional groups. The acrylate adhesive may alternatively be an acrylic adhesive having one or more —COOH functional groups. As such, non-functionalized pressure sensitive adhesives do not include one or more —COOH functional groups, such as found in the following pressure sensitive adhesives: Duro-Tak® 87-387, Duro-Tak® 87-2979 and Duro-Tak® 87-2353. Where the acrylic adhesive is non-functionalized, in some instances the pressure sensitive adhesive may be a composition that is or is substantially the same as, the composition of Duro-Tak® 87-9301.

Where the pressure sensitive adhesive includes polybutene, the polybutene may be saturated polybutene. Alternatively, the polybutene may be unsaturated polybutene. Still further, the polybutene may be a mixture or combination of saturated polybutene and unsaturated polybutene. In some embodiments, the pressure sensitive adhesive may include a composition that is, or is substantially the same as, the composition of Indopol® L-2, Indopol® L-3, Indopol® L-6, Indopol® L-8, Indopol® L-14, Indopol® H-7, Indopol® H-8, Indopol® H-15, Indopol® H-25, Indopol® H-35, Indopol® H-50, Indopol® H-100, Indopol® H-300, Indopol® H-1200, Indopol® H-1500, Indopol® H-1900, Indopol® H-2100, Indopol® H-6000, Indopol® H-18000, Panalane® L-14E, Panalane® H-300E and combinations thereof. In certain embodiments, the polybutene pressure-sensitive adhesive is Indopol® H-1900. In other embodiments, the polybutene pressure-sensitive adhesive is Panalane® H-300E.

The amount of pressure sensitive adhesive in the matrix may vary, where in some instances the amount of pressure sensitive adhesive in the matrix ranges from 0.1 mg to 2000 mg, such as 0.5 mg to 1500 mg, such as 1 to 1000 mg, such as 10 to 750 mg, and including 10 mg to 500 mg. As such, the amount of pressure sensitive adhesive in the matrix ranges in some instances from 1% to 99% w/w, such as 5% to 95% w/w, such as 10% to 95%, such as 15% to 90% w/w and including 20% to 85% w/w. In other embodiments, the amount of pressure sensitive adhesive in the subject transdermal compositions is 70% by weight or greater of the total weight of the transdermal composition, such as 75% by weight or greater, such as 80% by weight or greater, such as 85% by weight or greater, such as 90% by weight or greater of the total weight of the transdermal composition. The weight ratio of pressure sensitive adhesive to naloxone active agent in the subject compositions may vary, ranging in some instances from 4:1 to 1000 to 1, such as from 5:1 to 950:1, such as from 6:1 to 900:1, such as from 7:1 to 850:1, such as from 8:1 to 800:1, such as from 9:1 to 750:1 and including from 10:1 to 500:1.

In some embodiments, the matrix may further include one or more crosslinked polymers. When present, crosslinked polymers may perform a variety of different functions, where such functions may include, but are not limited to: drug crystallization inhibition, improvement of the duration of wear, and improvement of one or more physical properties, e.g., cold flow, tack, cohesive strength, of the adhesive, etc.

Crosslinked polymers that may be present in the matrix may vary. For example, the matrix may include an amine-containing hydrophilic polymer. Amine-containing polymers may include, but are not limited to, polyethyleneimine, amine-terminated polyethylene oxide, amine-terminated polyethylene/polypropylene oxide, polymers of dimethyl amino ethyl methacrylate, and copolymers of dimethyl amino ethyl methacrylate and vinyl pyrrolidone. In certain embodiments, the crosslinked polymer is crosslinked polyvinylpyrrolidone, such as for example PVP-CLM, PVP-CLM, PVP K17, PVP K30, PVP K90, etc.

Crosslinked polymers that may be present in the matrix also include crosslinked acrylic acid polymers. When present, the crosslinked acrylic acid polymer may vary. In certain aspects, the crosslinked acrylic acid polymer is a copolymer that includes acrylic acid monomers and non-acrylic acid monomers in any desired proportion. In other aspects, the crosslinked acrylic acid polymer is a crosslinked acrylic acid homopolymer, in which the polymer includes acrylic acid monomers and does not include non-acrylic acid monomers. Such polymers have the general formula:

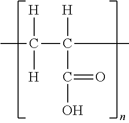

Crosslinked acrylic acid polymers of interest include carbomer polymers, which are high molecular weight, cross-linked non-linear polyacrylic acid polymers. Carbomer polymers of interest include, but are not limited to, the commercially available Carbopol® polymers (Lubrizol Corp., Wickliffe, Ohio). Carbopol® polymers that find use in the active agent layer of the compositions of the present disclosure include, e.g., Carbopol® 934P, Carbopol® 974P, Carbopol® 71G, Carbopol® 971P, Carbopol® 980, Carbopol® 981, Carbopol® 5984, Carbopol® ETD 2020, Carbopol® Ultrez 10, Carbopol® 934, Carbopol® 940, Carbopol® 941, Carbopol® 1342, or any combination thereof. According to certain embodiments, the crosslinked acrylic acid polymer is an acrylic acid homopolymer cross-linked with allyl ethers of sucrose. In certain aspects, an acrylic acid homopolymer cross-linked with allyl ethers of sucrose may have an average of from 4 to 6 allyl groups per sucrose molecule. For example, the crosslinked acrylic acid polymer may be Carbopol® 974P polymer. In other aspects, the crosslinked acrylic acid polymer is an acrylic acid homopolymer cross-linked with allyl ethers of pentaerythritol. Such acrylic acid homopolymers cross-linked with allyl ethers of pentaerythritol that may be present in the active agent layer of the subject transdermal compositions include, but are not limited to, Carbopol® 974P polymer. According to certain embodiments, the crosslinked acrylic acid polymer is an acrylic acid homopolymer crosslinked with divinyl glycol. For example, acrylic acid polymers crosslinked with divinyl glycol that may be present in the first active agent layer include, but are not limited to, Noveon® AA-1 Polycarbophil polymer (Lubrizol Corp., Wickliffe, Ohio). While the molecular weight of the crosslinked acrylic acid polymers may vary, in some instances the molecular weight ranges from 1,000 to 100,000,000, such as 3,000 to 10,000,000, and including 10,000 to 5,000,000.

The amount of crosslinked polymer in the matrix may vary, ranging in some instances from 0.1 mg to 500 mg, such as 0.5 mg to 400 mg, such as 1 to 300 mg, such as 10 to 200 mg, and including 10 mg to 100 mg. The amount of crosslinked polymer in the matrix ranges, in some instances, from 2% to 30% w/w, such as 4% to 30% w/w, such as 5% to 25%, such as 6% to 22.5% w/w and including 10% to 20% w/w. In some instances, the amount of crosslinked polymer in the matrix is 8% by weight or greater of the total weight of the transdermal composition, such as 10% by weight or greater, such as 12% by weight or greater, such as 15% by weight or greater, such as 20% by weight or greater, such as 25% by weight or greater and including 30% by weight crosslinked polymer or greater of the total weight of the transdermal composition.

In certain embodiments, the matrix further includes a skin penetration enhancer. By “skin penetration enhancer” is meant a compound or composition which increases flux of drug across skin compared to a reference solution at the same constant fraction of drug saturation. In some embodiments, the skin penetration enhancers increase the flux of the active agent through the skin by 5% or more as compared to a reference composition having the same constant fraction of drug saturation, such as by 10% or more, such as by 15% or more, such as by 20% or more, such as by 25% or more, such as by 30% or more, such as by 35% or more, such as by 50% or more, such as by 75% or more and including by 90% or more.

Skin penetration enhancers that may be present in the matrix include, but are not limited to, fatty acids, e.g., saturated fatty acids, such as C8 to C16 saturated fatty acids, e.g., caprylic acid, capric acid, lauric acid, myristic acid, palmitic acid, etc.; unsaturated fatty acids such as C8 to C18 unsaturated fatty acids, including linoleic acid, oleic acid, linolenic acid, etc., as well as other acids, e.g., levulinic acid, N-lauroyl sarcosine, L-pyroglutamic acid, succinic acid, pyruvic acid, glutaric acid, sebacic acid, cyclopentane carboxylic acid; acylated amino acids, etc. Other skin penetration enhances that may be present in the matrix include, but are not limited to, aliphatic alcohols, such as saturated or unsaturated higher fatty alcohols having 10 to 22 carbon atoms (e.g., oleyl alcohol or lauryl alcohol); fatty acid esters, such as isopropyl myristate, diisopropyl adipate, lauryl lactate, propylene glycol monolaurate, propyl laurate, ethyl oleate, and isopropyl palmitate; alcohol amines, such as triethanolamine, triethanolamine hydrochloride, and diisopropanolamine; polyhydric alcohol alkyl ethers, such as alkyl ethers of polyhydric alcohols such as glycerol, ethylene glycol, propylene glycol, 1,3-butylene glycol, diglycerol, hexylene glycol, polyglycerol, diethylene glycol, polyethylene glycol, dipropylene glycol, polypropylene glycol, polypropylene glycolmonolaurate, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as polyoxyethylene alkyl ethers in which the number of carbon atoms of the alkyl group moiety is 6 to 20, and the number of repeating units (e.g. —O—CH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone; pyrrolidone derivatives and combinations thereof. Additional types of skin penetration enhancers may include lactic acid, tartaric acid, 1,2,6-hexanetriol, benzyl alcohol, lanoline, potassium hydroxide (KOH), tris(hydroxymethyl)aminomethane, glycerol monooleate (GMO), sorbitan monolaurate (SML), sorbitan monooleate (SMO), laureth-4 (LTH), and combinations thereof. In some embodiments, the penetration enhancer is a combination of a surfactant enhancer with a solvent enhancer such as a semi-polar liquid, where semi-polar liquids include, but are not limited to, propylene glycol, butanediol, or DMSO. The amount of each skin penetration enhancer present in the matrix may vary, and in some instances ranges from 1 to 25% (w/w), such as from 1 to 15% (w/w), such as from 2 to 12.5% (w/w), such as from 3 to 10% (w/w), such as from 4 to 8% (w/w) and including from 1 to 10% (w/w). Where the skin penetration enhancer is a combination of two or more enhancers, such as a combination of a surfactant enhancer with a solvent enhancer, the total amount of the skin penetration enhancer component in naloxone compositions of interest may range from 1% to 50% (w/w), such as from 2% to 45% (w/w), such as from 3% to 40% (w/w), such as from 4% to 35% (w/w) and including from 5% to 25% (w/w).

The size of naloxone transdermal delivery devices may vary. As such, the transdermal delivery device may have a surface area ranging from 4 cm² to 10,000 cm², such as from 5 cm² to 1000 cm², such as from 10 cm² to 100 cm², such as from 15 cm² to 50 cm² and including from 20 cm² to 40 cm².

In addition to the matrix, the transdermal delivery devices, e.g., as described above, further include a backing layer. The backing layer may be flexible, so that it can be brought into close contact with the desired application site on the subject. The backing layer may be fabricated from a material that does not absorb the naloxone active agent, and does not allow the naloxone active agent to be leached from the matrix. Backing layers of interest that may be employed in transdermal delivery devices of the invention include, but are not limited to, non-woven fabrics, woven fabrics, films (including sheets), porous bodies, foamed bodies, paper, composite materials obtained by laminating a film on a non-woven fabric or fabric, and combinations thereof. In some instances, the backing layer is a non-metallic backing layer.

Non-woven fabrics may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabrics may include cotton, rayon, polyacrylic resins, polyester resins, polyvinyl alcohol, and combinations thereof. Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. Papers may include impregnated paper, coated paper, wood free paper, Kraft paper, Japanese paper, glassine paper, synthetic paper, and combinations thereof.

The size of the backing layer may vary, and in some instances the backing layer is sized to cover the entire application site on the subject. As such, the backing layer may have a length ranging from 2 to 100 cm, such as 4 to 60 cm and a width ranging from 2 to 100 cm, such as 4 to 60 cm. In certain instances, the backing layer may be insoluble in water. By insoluble in water is meant that that the backing layer may be immersed in water for a period of 1 day or longer, such as 1 week or longer, including 1 month or longer, and exhibit little if any dissolution, e.g., no observable dissolution. Transdermal delivery devices according to embodiments of the invention are those that have an acceptably low severity and incidence of skin irritation at the application site, and in some instances are non-irritating to the skin of the subject at the site of application.

Irritation of the skin is referred to herein in its general sense to refer to adverse effects, discoloration or inflammation of the skin, such as for example, redness, pain, itching, swelling, dryness, or eschars. As such, in practicing methods with the subject transdermal delivery devices, the quality of the skin remains normal (e.g., when visually inspected with the naked eye or when aided with a visualization device), and transdermal delivery is consistent throughout the entire dosage interval.

In some embodiments, skin irritation is evaluated to determine the quality and color of the skin at the application site and to determine whether any damage, pain, swelling or dryness has resulted from maintaining the transdermal composition in contact with the subject. The skin may be evaluated for irritation, sensitization, and other dermatological conditions in man or an appropriate animal model by any convenient protocol, such as for example using the Draize scale, as disclosed in Draize, J. H., Appraisal of the Safety of Chemicals in Foods, Drugs and Cosmetics, pp. 46-49, The Association of Food and Drug Officials of the United States: Austin, Tex. or in Marzulli, F N, and Maibach, H I. (1983) Dermatotoxicology 2^{nd} ed. Hemisphere Publishing Corp, Weltfriend, s, Bason, D, Lammintausta, K, and Maibach, H I. (1996) “Irritant Dermatitis.” in Marzulli, F N, and Maibach, H I (eds). “Dermatotoxicology”, 5th ed. pp. 87-118.

Taylor & Francis, Wash, D.C.; FDA CDER (December, 1999). “Skin Irritation and Sensitization Testing of Generic Transdermal Products, the disclosure of which is herein incorporated by reference. In particular, the skin may be evaluated at the transdermal application site for erythema or edema. For example, grades for erythema and edema may be assigned based on visual observation or palpation:
Erythema: 0=no visible redness; 1=very slight redness (just perceptible); 2=slight but defined redness; 3=moderately intense redness; 4=severe erythema (dark red discoloration of the skin)
   5=eschar formation
Edema: 0=no visible reactions or swelling; 1=very mild edema (just perceptible swelling); 2=mild edema (corners of area are well defined due to swelling); 3=moderate edema (up to 1 mm swelling); 4=severe edema (more than 1 mm swelling).

In other embodiments, the site of transdermal application is evaluated for skin irritation after the transdermal delivery device has been removed from contact with the subject. For example, the site of application may be evaluated for skin irritation 30 minutes after removing the transdermal delivery device, such as 1 hour after removing the transdermal delivery device, such as 2 hours after removing the transdermal delivery device, such as 4 hours after removing the transdermal delivery device, such as 8 hours after removing the transdermal delivery device, such as 12 hours after removing the transdermal delivery device, such as 24 hours after removing the transdermal delivery device, such as 48 hours after removing the transdermal delivery device, including 72 hours after removing the transdermal delivery device.

In some embodiments, the site of transdermal application is evaluated for skin irritation before the transdermal delivery device is applied to a subject, such as to record the skin color and texture before commencing a dosage interval. For example, the site of application may be evaluated for skin irritation 5 minutes before applying the transdermal delivery device, such as 10 minutes, such as 30 minutes, such as 60 minutes, such as 120 minutes, such as 240 minutes and including 480 minutes before applying the transdermal delivery device. Where methods include multiple dosage intervals applied sequentially, the site of application may be evaluated for skin irritation after each transdermal delivery device is removed and before the subsequent transdermal delivery device is applied. For example, when a first transdermal delivery device is removed, the site of application may be evaluated for skin irritation 2 hours, 24 hours and 48 hours after removal and before application of a second transdermal delivery device. A subsequent transdermal delivery device may be applied to the previous site of application or a different location on the body immediately after evaluating the skin for irritation or may be applied after a predetermined time after evaluating the skin for irritation, such as 1 hour, 4 hours, 12 hours, 24 hours, 48 hours, 72 hours, 96 hours, 120 hours, 144 hours or 168 hours after evaluating the skin for irritation. As desired, the application site may be rotated to another skin site for each successive patch application. In certain testing of irritation or sensitization, the transdermal patch may be applied successively to the same skin site for a determined number of times or total duration.

The site of application may be evaluated for skin irritation one or more times before, during or after a dosage interval, such as 2 or more times, such as 3 or more times, including 5 or more times before, during or after a dosage interval. An upper limit for the number of times the site of application may be evaluated for skin irritation before, during or after a dosage interval is, in some instances, 10 times or fewer, such as 7 times or fewer, such as 5 times or fewer, such as 3 times or fewer and including 2 times or fewer. In certain embodiments, the number of times the site of application may be evaluated for skin irritation before, during or after a dosage interval ranges such as from 2 times to 10 times, such as from 3 times to 9 times, such as from 4 times to 8 times and including from 5 times to 7 times. In certain embodiments, skin irritation may be monitored throughout the entire time the transdermal delivery device is maintained in contact with the subject, such by video monitoring.

In some embodiments, the transdermal compositions of the present disclosure include a release liner. The release liner may be disposed directly on the matrix and removed prior to use. The release liner facilitates the protection of the matrix of the transdermal delivery device prior to use. In certain aspects, a release liner may be prepared by treating one side of polyethylene-coated wood free paper, polyolefin-coated glassine paper, a polyethylene terephthalate (polyester) film, a polypropylene film, or the like with a silicone treatment.

Optionally, one or more adhesive overlays can be used to increase the adhesion of the composition when applied to the skin. Adhesive overlays can include a layer of adhesive present on a backing material, such as a porous, non-porous, occlusive, or breathable backing material. The dimensions of the adhesive overlay are chosen to provide the desired functionality, where in some instances the dimensions are chose such that the adhesive overlay, when applied over the active agent formulation, extends some distance beyond one or more of the sides of the active agent formulation. In some instances, the area of the adhesive overlay exceeds the area of the active agent layer by 5% or more, such as by 10% or more, such as by 20% or more, such as by 30% or more, such as by 40% or more, such as by 50% or more, such as by 60% or more, such as by 70% or more, such as by 80% or more, such as by 90% or more, such as by 95% or more, such as by 99% or more and including by 100% or more. During use, the adhesive overlay can be applied by the patients, by the care givers, can be integrated into the patch itself, or can be integrated in the kits.

### Methods for Applying Naloxone Transdermal Delivery Devices

Aspects of the invention also include methods for applying naloxone transdermal delivery devices to a subject. The term “transdermal” is used in its conventional sense to refer to the route of administration where an active agent (i.e., drug) is delivered across the skin or mucous membrane for systemic distribution. As such, transdermal naloxone compositions as described herein include compositions which are delivered to the subject through one or more of the subcutis, dermis and viable epidermis, including the stratum corneum, stratum germinativum, stratum spinosum and stratum basale into the capillaries that penetrate and underlay the dermis or approach the epidermis. Accordingly, extended transdermal delivery devices containing a transdermal naloxone composition may be applied at any convenient location, such as for example, the arms, legs, buttocks, chest, abdomen, back, thigh neck, scrotum, vagina, face, behind the ear, buccally as well as sublingually. In certain embodiments, the naloxone transdermal patch is applied to one or more of the arms, back, buttocks, abdomen, and thighs. In some embodiments, the skin is healthy intact skin. In other embodiments, the skin may be skin where one or more layers (e.g., stratum corneum, stratum germinativum, stratum spinosum, stratum basale, etc.) may be diseased, inflamed or not fully intact. The phrase “not fully intact” is used herein in its conventional sense to mean that the one or more layers of not fully intact skin includes at least one perforation resulting from disease, inflammation or other condition, where not fully intact skin may include perforations that is cumulatively 0.01% or more of the surface area where the topical naloxone composition is applied, such as 0.05% or more, such as 0.1% or more, such as 0.5% or more, such as 1% or more, such as 2% or more, such as 3% or more, such as 5% or more, such as 10% or more, such as 25% or more, such as 50% or more, such as 75% or more, such as 90% or more, such as 95% or more, such as 97% or more and including 99% or more of the surface area where the naloxone transdermal delivery device is applied.

In describing methods of the present invention, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). As such, subjects of the invention, include but are not limited to mammals, e.g., humans and other primates, such as chimpanzees and other apes and monkey species; and the like, where in certain embodiments the subject are humans. The term subject is also meant to include a person or organism of any age, weight or other physical characteristic, where the subjects may be an adult, a child, an infant or a newborn.

In some embodiments, methods include extended transdermal delivery of naloxone to the subject. As described above, by “extended transdermal delivery” is meant that transdermal administration is formulated to provide for delivery of the naloxone to the subject over an extended period of time, such as over the course of hours, days and including weeks, including 1 hour or longer, such as 2 hours or longer, such as 4 hours or longer, such as 8 hours or longer, such as 12 hours or longer, such as 24 hours (1 day) or longer, such as 48 hours (2 days) or longer, such as 72 hours (3 days) or longer, such as 96 hours (4 days) or longer, such as 120 hours (5 days) or longer, such as 144 hours (6 days) or longer and including 168 hours (7 days) or longer. For the above ranges an upper limit period of time is, in some instances, 168 hours or shorter, such as 144 hours or shorter, such as 120 hours or shorter, such as 96 hours or shorter, such as 72 hours or shorter, such as 48 hours or shorter and including 24 hours or shorter. In certain embodiments, extended transdermal delivery ranges from 0.5 hours to 168 hours, such as from 1 hour to 144 hours, such as from 1.5 hours to 120 hours, such from 2 hours to 96 hours, such as from 2.5 hours to 72 hours, such as from 3 hours to 48 hours, such as from 3.5 hours to 24 hours, such as from 4 hours to 12 hours and including from 5 hours to 8 hours.

In some embodiments, sustained release transdermal administration of the naloxone includes multi-day delivery of a therapeutically effective amount of naloxone to the subject. By multi-day delivery is meant that the transdermal composition is formulated to provide a therapeutically effective amount of naloxone to a subject when the transdermal delivery device is applied to the skin of a subject for a period of time that is 1 day or longer, such as 2 days or longer, such as 4 days or longer, such as 7 days or longer. In certain embodiments, transdermal delivery devices provide a therapeutically effective amount of naloxone to a subject for a period of 2 days or longer, such as 3 days or longer. For multi-day delivery, an upper limit period of time is, in some instances, 10 days or shorter, such as 7 days or shorter. In certain embodiments, multi-day transdermal delivery ranges such as from 2 days to 10 days, such as from 3 days to 7 days.

As described above, aspects of the invention include applying a naloxone transdermal delivery device to a subject and maintaining the matrix of the applied device at the application site of the subject for a period of time sufficient to deliver naloxone to the subject. In some embodiments, the methods include maintaining the matrix of the transdermal delivery device in contact with a subject in a manner sufficient to deliver a target dosage of naloxone to the subject, such as for example delivering a target dosage as determined by total drug exposure or by average daily drug exposure. Depending on the desired therapeutic effect of the naloxone transdermal delivery device, the treatment protocol and the physiology of the subject, target drug exposure may vary. In certain embodiments, the target drug exposure of naloxone that results by practice of the methods is an amount which is in the therapeutic window of the subject. In embodiments of the invention, a therapeutically effective amount provides for a systemic amount of naloxone that enables the desired treatment. The dosage delivered by a given naloxone transdermal delivery device may vary, where in some instances the delivered dosage of naloxone ranges from 0.1 μg/day to 10,000 μg/day, such as from 1 μg/day to 8,000 μg/day, including from 50 μg/day to 5,000 μg/day, e.g., from 100 μg/day to 3,000 μg/day. In some embodiments, the delivered or target dosage is an amount that provides for a systemic amount of naloxone that results in a desired mean plasma concentration of naloxone at specific times during treatment. In other embodiments, the delivered or target dosage is an amount that when applied to a subject provides for a steady state mean plasma concentration of naloxone throughout a dosage interval or treatment protocol. By “steady state mean plasma concentration” is meant that the plasma concentration of naloxone remains constant through the duration of the treatment interval or protocol, such as where the mean plasma concentration of naloxone changes (i.e., increase or decrease) by 50% or less, such as by 45% or less, such as by 40% or less, such as by 35% or less, such as by 30% or less, such as by 25% or less, such as by 20% or less, such as by 15% or less, such as by 10% or less, such as by 5% or less, such as by 4% or less, such as by 3% or less such as by 2% or less, such as by 1% or less and including by 0.1% or less. In other embodiments, the delivered or target dosage is an amount that when applied to a subject provides for a particular rate of delivery of naloxone to the subject.

In some embodiments, the delivered dosage provides for an average cumulative systemic amount of naloxone over the course of a dosage interval (e.g., 1 day or longer, such as 3 days or longer). As noted above, the term “average cumulative systemic amount” is used to refer to the total quantity of naloxone which is delivered to the subject through the skin and is absorbed systemically. In some embodiments, the average cumulative systemic amount of delivered naloxone may be 0.01 μg/cm² or greater, such as 0.1 μg/cm² or greater, e.g., 1 μg/cm² or greater, including 10 μg/cm², and in some instances up to 50 μg/cm² or greater over a dosage interval, such as a 1, 3 or 7 day dosage interval. In some embodiments, the amounts of naloxone delivered across skin in vivo can be determined by in vitro skin flux experiments, such as by the difference in residual drug extracted from worn and unused patches, or from the product of the AUC extrapolated to infinity and the clearance.

In other embodiments, the delivered dosage is an amount that provides for a local amount of naloxone that results in a desired therapeutic activity. In these embodiments, the delivered amount is an amount of naloxone that provides for an average cumulative local amount of naloxone delivered over the course of a dosage interval (e.g., 1 day or longer, such as 3 days or longer). The term “average cumulative local amount” is used to refer to the total quantity of naloxone which is delivered locally to the subject through the skin. In some embodiments, the average cumulative local amount of delivered naloxone may be 0.01 μg/cm² or greater, such as 0.1 μg/cm² or greater, e.g., 1 μg/cm² or greater, including 10 μg/cm², and in some instances up to 50 μg/cm² or greater over a dosage interval, such as a 1, 3 or 7 day dosage interval.

In certain embodiments, methods may also include determining the plasma concentration of naloxone in the subject. The plasma concentration may be determined using any convenient protocol, such for example by liquid chromatography-mass spectrometry (LCMS). The plasma concentration of the naloxone may be determined at any time desired. In some embodiments, the plasma concentration of naloxone may be monitored throughout the entire time the transdermal delivery device is maintained in contact with the subject, such by real-time data collection. In other instances, the plasma concentration of naloxone is monitored while maintaining the transdermal delivery device in contact with the subject by collecting data at regular intervals, e.g., collecting data every 0.25 hours, every 0.5 hours, every 1 hour, every 2 hours, every 4 hours, every 12 hours, every 24 hours, including every 72 hours, or some other interval. In yet other instances, the plasma concentration of naloxone is monitored while maintaining the transdermal delivery device in contact with the subject by collecting data according to a particular time schedule after applying the transdermal delivery device to the subject. For instance, the plasma concentration of naloxone may be determined 15 minutes after applying the transdermal delivery device to the subject, 30 minutes after applying the transdermal delivery device to the subject, 1 hour after applying the transdermal delivery device to the subject, 2 hours after applying the transdermal delivery device to the subject, 4 hours after applying the transdermal delivery device to the subject, 8 hours after applying the transdermal delivery device to the subject, 12 hours after applying the transdermal delivery device to the subject, 24 hours after applying the transdermal delivery device to the subject, 48 hours after applying the transdermal delivery device to the subject, 72 hours after applying the transdermal delivery device to the subject, 76 hours after applying the transdermal delivery device to the subject, 80 hours after applying the transdermal delivery device to the subject, 84 hours after applying the transdermal delivery device to the subject, 96 hours after applying the transdermal delivery device to the subject, 120 hours after applying the transdermal delivery device to the subject and including 168 hours after applying the transdermal delivery device to the subject.

In certain embodiments, the plasma concentration of naloxone is determined before the transdermal delivery device is applied to a subject, such as for example, to determine the basal plasma concentration of the naloxone. For example, the plasma concentration may be determined 5 minutes before applying the transdermal delivery device, such as 10 minutes before, such as 30 minutes before, such as 60 minutes before, such as 120 minutes before, such as 240 minutes before and including 480 minutes before applying the transdermal delivery device. As described detail below, methods may include multiple dosage intervals where applying and maintaining the transdermal delivery device in contact with the subject may be repeated. In these embodiments, the plasma concentration may be determined after a first transdermal delivery device is removed and before a second transdermal delivery device is applied.

The blood plasma concentration of the naloxone may be determined one or more times at any given measurement period, such as 2 or more times, such as 3 or more times, including 5 or more times at each measurement period. An upper limit for the number of times the blood plasma concentration of naloxone is determined at any given measurement period is, in some instances, 10 times or fewer, such as 7 times or fewer, such as 5 times or fewer, such as 3 times or fewer and including 2 times or fewer. In certain embodiments, the number of times the blood plasma concentration of naloxone is determined at any given measurement period ranges such as from 2 times to 10 times, such as from 3 times to 9 times, such as from 4 times to 8 times and including from 5 times to 7 times.

Methods according to certain embodiments may include applying to the subject a naloxone transdermal delivery device and maintaining the naloxone transdermal delivery device in contact with the subject in a manner sufficient to maintain a transdermal naloxone flux which is within 5 to 150% of the peak transdermal naloxone flux after reaching the peak transdermal flux. As such, once transdermal delivery devices of interest reach peak transdermal naloxone flux, the transdermal delivery device is configured to maintain a flux of naloxone to the subject that is from 5 to 150% of peak flux during the course of any given dosage interval, such as from 10% to 145%, such as from 15% to 140%, such as from 20% to 135%, such as from 25% to 130%, such as from 30% to 125%, such as from 35% to 120%, such as from 40% to 115%, such as from 45% to 110%, such as from 50% to 105%, such as from 55% to 100%, such as from 60% to 95% and including from 65% to 90%. For example, the naloxone transdermal device may be maintained in contact with the subject in a manner sufficient to maintain the transdermal naloxone flux which is 80% or more of peak transdermal naloxone flux, such as 85% or more, such as 90% or more, such as 95% and including 99% of peak transdermal naloxone flux after reaching peak transdermal flux. In certain embodiments, the transdermal naloxone flux does not decrease at all after reaching peak flux and maintains a rate of 100% of peak naloxone flux from the moment it reaches peak flux until the end of a given dosage interval.

The flux of an active agent by transdermal administration is the rate of penetration of the active agent through the skin or mucous membrane of the subject. In some instances, the flux of naloxone can be determined by the equation:

J_{skin flux}=P×C (1)

where J is the skin flux, C is the concentration gradient across the skin or mucous membrane and P is the permeability coefficient. Skin flux at a given time is the first derivative in cumulative amount of drug entering the body across the skin or mucous membrane per unit area with respect to time.

In some instances, the naloxone transdermal delivery device is maintained in contact with the subject in a manner sufficient to provide a peak flux of 10 g/cm²/hr or less, such as 9 μg/cm²/hr or less, such as 8 μg/cm²/hr or less, such as 7 μg/cm²/hr or less, such as 6 μg/cm²/hr or less, such as 5 μg/cm²/hr or less, such as 4 μg/cm²/hr or less, such as 3 μg/cm²/hr or less, such as 2 μg/cm²/hr or less, such as 1 μg/cm²/hr or less, such as 0.5 μg/cm²/hr or less and including 0.1 g/cm²/hr or less. In some embodiments, the naloxone transdermal delivery device is maintained in contact with the subject in a manner sufficient to provide a peak flux of from 0.01 μg/cm²/hr to 15 μg/cm²/hr, such as from 0.05 μg/cm²/hr to 14 μg/cm²/hr, such as from 0.1 μg/cm²/hr to 13 μg/cm²/hr, such as from 0.5 g/cm²/hr to 12 μg/cm²/hr, such as from 1 μg/cm²/hr to 11 μg/cm²/hr, such as from 2 μg/cm²/hr to 10 μg/cm²/hr, such as from 3 μg/cm²/hr to 9 μg/cm²/hr and including from 4 μg/cm²/hr to 8 μg/cm²/hr. Depending on the amount of naloxone present in the matrix of the naloxone transdermal delivery device, the physiology of the subject and the target site of application, the time required to reach peak naloxone flux may vary. In some instances, peak naloxone flux is reached 2 hours or more after applying the transdermal delivery device to the subject, such as 4 hours or more, such as 6 hours or more, such as 12 hours or more, such as 18 hours or more and including at 24 hours or more after applying the transdermal delivery device to the subject. In other instances, the peak naloxone flux is reached at 168 hours or earlier, such as 144 hours or earlier, such as 120 hours or earlier, such as 96 hours or earlier, such as 72 hours or earlier, such as 48 hours or earlier, such as 24 hours or earlier, such as 12 hours or earlier, such as 8 hours earlier, such as 4 hours or earlier and including at 2 hours or earlier. In some embodiments, peak naloxone flux is reached at 24 hours after applying the transdermal delivery device to the subject.

In certain embodiments, the matrix of the transdermal delivery device is maintained in contact with the subject sufficient to provide a steady state average flux of naloxone to the subject. As such, the naloxone flux from transdermal delivery devices of interest increases or decreases by 30% or less at any time while the transdermal delivery device is maintained in contact with the subject following an initial post-application period (such as described above), such as 20% or less, such as 15% or less, such as 12% or less, such as 10% or less, such as 6% or less, such as 5% or less, such as 4% or less, and including 1% or less at any time while the transdermal delivery device is maintained in contact with the subject. In some instances, the steady state average naloxone flux may be maintained from for 0.5 hours or longer, such as 1 hour or longer, such as 2 hours or longer, such as 3 hours or longer, such as 4 hours or longer, such as 8 hours or longer, 12 hours or longer, such as 24 hours or longer, such as 36 hours or longer, such as 48 hours or longer, such as 72 hours or longer, such as 96 hours or longer, such as 120 hours or longer, such as 144 hours or longer and including 168 hours or longer. For maintaining a steady state average naloxone flux, an upper limit is, in some instances, for 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter. In these embodiments, the transdermal delivery device is configured to provide a constant flux, such as by introducing a concentration gradient across the skin or mucous membrane or providing an excess in naloxone dosage amount. For example, the matrix may include a naloxone dosage that is 5% or greater in excess of the normal dosage amount, such as 10% or greater, such as 15% or greater, such as 20% or greater, and including 25% or greater in excess of the normal dosage amount. For amount of excess naloxone present in the transdermal delivery device to provide a constant flux, an upper limit is, in some instances 50% or less in excess, such as 45% or less in excess, such as 25% or less in excess, such as 20% or less in excess and including 10% or less in excess of the normal dosage amount. While the matrix may include an excess in order to provide a constant flux, the excess dosage amount is not absorbed as part of the dosage interval. As such, in some embodiments where the naloxone transdermal delivery device is maintained in a manner sufficient to provide a constant flux, 25% or less of the available naloxone in the transdermal composition may not be utilized, such as 20% or less, such as 15% or less, such as 10% or less, such as 5% or less and including 1% or less of the available naloxone in the transdermal composition may not be utilized during the dosage interval.

Methods according to certain embodiments may include applying to the subject a naloxone transdermal delivery device and maintaining the applied naloxone transdermal delivery device in contact with the subject in a manner sufficient to provide an average flux of naloxone of from 0.001 to 15 μg/cm²/hr, such as from 0.005 μg/cm²/hr to 14 μg/cm²/hr, such as from 0.01 μg/cm²/hr to 13 g/cm²/hr, such as from 0.05 μg/cm²/hr to 12 μg/cm²/hr, such as from 0.1 g/cm²/hr to 11 μg/cm²/hr, such as from 0.5 μg/cm²/hr to 10 μg/cm²/hr, such as from 1 μg/cm²/hr to 9 μg/cm²/hr and including from 2 μg/cm²/hr to 8 μg/cm²/hr at any time after applying the transdermal delivery device.

In certain embodiments, methods include determining the transdermal naloxone flux. The transdermal naloxone flux may be determined using any convenient, well-designed protocol, such for example by protocols employing human cadaver skin with epidermal layers (stratum corneum and epidermis) in a Franz cell, flow through cell, etc. The amount of permeated naloxone can further be characterized by liquid chromatography by sampling at selected times. The transdermal naloxone flux may be determined at any time during methods of the invention. In some embodiments, the transdermal naloxone flux may be monitored throughout the entire time the naloxone transdermal delivery device is maintained in contact with the permeation barrier (e.g., human cadaver skin), such by real-time data collection. In other instances, the naloxone flux is monitored by collecting data at regular intervals, e.g., collecting data every 0.25 hours, every 0.5 hours, every 1 hour, every 2 hours, every 4 hours, every 12 hours, every 24 hours, including every 72 hours, or some other regular or irregular intervals. In yet other instances, the naloxone flux is monitored by collecting data according to a particular time schedule. For instance, naloxone flux may be determined 15 minutes after applying the transdermal delivery device, 30 minutes after applying the transdermal delivery device, 1 hour after applying the transdermal delivery device, 2 hours after applying the transdermal delivery device, 4 hours after applying the transdermal delivery device, 8 hours after applying the transdermal delivery device, 12 hours after applying the transdermal delivery device, 24 hours after applying the transdermal delivery device, 48 hours after applying the transdermal delivery device, 72 hours after applying the transdermal delivery device, 76 hours after applying the transdermal delivery device, 80 hours after applying the transdermal delivery device, 84 hours after applying the transdermal delivery device, 96 hours after applying the transdermal delivery device, 120 hours after applying the transdermal delivery device and including 168 hours after applying the transdermal delivery device. The naloxone flux may be determined one or more times at any given measurement period, such as 2 or more times, such as 3 or more times, including 5 or more times at each measurement period. An upper limit for the number of times the transdermal naloxone flux is determined is, in some instances, 10 times or fewer, such as 7 times or fewer, such as 5 times or fewer, such as 3 times or fewer and including 2 times or fewer. In certain embodiments, the number of times the naloxone flux is determined ranges such as from 2 times to 10 times, such as from 3 times to 9 times, such as from 4 times to 8 times and including from 5 times to 7 times.

In some embodiments, in maintaining the naloxone transdermal delivery device in contact with the subject, the average cumulative amount of permeated naloxone increases at a substantially linear rate over the course of the dosage interval (e.g., 7 days or longer). By “substantially linearly” is meant that the cumulative amount of naloxone released from matrix increases at a substantially constant rate (i.e., defined by zero-order kinetics or steady-state flux). As such, the change in rate of cumulative permeated naloxone increases or decreases by 10% or less at any given time while maintaining the transdermal composition in contact with the subject, such as 8% or less, such as 7% or less, such as 6% or less, such as 5% or less, such as 3% or less, such as 2.5% or less, such as 2% or less, and including 1% or less at any time while maintaining the matrix of the transdermal delivery device in contact with the subject. In some instances, the cumulative permeated naloxone after the initial post-application time period increases linearly with time. In certain instances, the cumulative permeated naloxone increases more rapidly in an early phase due to a peak flux and after this period nearly linearly with time. In some instances, the cumulative permeated naloxone after a nearly linear increase with time proceeds to increase more slowly, such as for example due to depletion of drug or enhancer.

As described above, aspects of the invention include applying to a subject a naloxone transdermal delivery device and maintaining the matrix of the applied device in contact with the subject over a period of time sufficient to deliver naloxone to the subject, i.e., over a dosage interval. In some embodiments, methods may include maintaining the matrix of the applied transdermal delivery device in contact with the subject in a manner sufficient to deliver a predetermined amount of naloxone to the subject. Where protocols include delivering a predetermined amount of naloxone to the subject, the amount of naloxone in the matrix may vary, e.g., as described above. In certain embodiments, the predetermined amount of naloxone delivered to the subject may be a percentage of the total amount of naloxone present in the matrix. For instance, the predetermined amount of naloxone delivered to the subject may be 1% or greater of the total amount of naloxone present in the matrix, such as 2% or greater, such as 5% or greater, such as 10% or greater, such as 25% or greater and including 50% or greater of the total amount of naloxone present in matrix. In other words, the methods may include maintaining the matrix of the transdermal delivery device in contact with the subject in a manner sufficient to deliver 5% or greater of the naloxone in the matrix over the course of a single dosage interval. In these embodiments, the utilization percentage of naloxone is 5% or greater during the time the matrix is maintained in contact with the subject. As such, 95% or less of the original amount of naloxone remains in the matrix after a dosage interval. As described in greater detail below, the naloxone transdermal delivery devices are capable of high utilization percentage. In other words, the naloxone transdermal delivery devices are capable of delivering naloxone to the subject leaving little residual naloxone in the matrix. The utilization percentage may be 5% or greater over the course of a dosage interval, such as 10% or greater, such as 25% or greater, such as 40% or greater, such as 45% or greater and including 50% or greater of the naloxone over the course of a dosage interval. For utilization percentage, an upper limit over the course of a dosage interval is, in some instances, 90% or less, such as 50% or less, such as 25% or less and including 5% or less over the course of a dosage interval.

In certain embodiments, protocols may include multiple dosage intervals. By “multiple dosage intervals” is meant more than one transdermal delivery device is applied and maintained in contact with the subject in a sequential manner. As such, a transdermal delivery device is removed from contact with the subject and a new transdermal delivery device is reapplied to the subject. In some instances, sequential patches are applied to a different skin site than the immediately previous patch, such as for example to avoid skin irritation. In practicing methods of the invention, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 3 days or longer, such as 4 days or longer, such as 5 days or longer, such as 6 days or longer, such as 7 days or longer, such as 10 days or longer, including 30 days or longer. An upper limit period of time between dosage intervals is, in some instances, 30 days or shorter, such as 28 days or shorter, such as 21 days or shorter, such as 14 days or shorter, such as 7 days or shorter and including 3 days or shorter. In certain embodiments, the time between dosage intervals ranges such as from 2 days to 30 days, such as from 3 days to 28 days, such as from 4 days to 21 days, such as from 5 days to 14 days and including from 6 days to 10 days.

In certain instances, the duration between dosage intervals may depend on the plasma concentration of naloxone during the time the transdermal delivery device is not in contact with the subject between dosage intervals. For example, a subsequent dosage interval may commence when the plasma concentration of naloxone reaches below a particular threshold.

In certain embodiments, each of the subject methods described in greater detail below may further include the step of removing the transdermal delivery device from contact with the subject at the conclusion of a dosage interval. By “removing” the transdermal delivery device from contact with the subject is meant that no portion of the transdermal delivery device physically contacts the skin surface to which the device was applied. For example, the transdermal delivery device may be removed from contact with the subject after maintaining the transdermal delivery device in contact with the subject for 0.5 hours or more, such as 1 hour or more, such as 2 hours or more, such as 4 hours or more, such as 8 hours or more, such as 12 hours or more, such as 24 hours or more, such as 36 hours or more, such as 48 hours or more, such as 60 hours or more, such as 72 hours or more, such as 96 hours or more, such as 120 hours or more, including 144 hours or more, and including 168 hours or more. An upper limit for the amount of time the transdermal delivery device is maintained in contact with a subject before removal is, in some instances, 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter.

As described above, a dosage interval is a single administration of applying and maintaining the transdermal delivery device in contact with the subject which begins with applying the device to the topical application site, e.g., skin or mucous membrane of the subject, and ends with the removal of the transdermal delivery device from the topical location of the subject. Also as reviewed above, in certain embodiments, protocols may include multiple dosage intervals. By “multiple dosage intervals” is meant more than one transdermal delivery device is applied and maintained in contact with the subject in a sequential manner. As such, a transdermal delivery device is removed from contact with the subject and a new transdermal delivery device is reapplied to the subject. In practicing methods of the invention, treatment regimens may include two or more dosage intervals, such as three or more dosage intervals, such as four or more dosage intervals, such as five or more dosage intervals, including ten or more dosage intervals.

The location on the subject for reapplying subsequent transdermal delivery devices in multiple dosage treatment regimens may be the same or different from the location on the subject where the previous transdermal delivery device was removed. For example, if a first transdermal delivery device is applied and maintained on the leg of the subject, one or more subsequent transdermal delivery devices may be reapplied to the same position on the leg of the subject. On the other hand, if a first transdermal delivery device was applied and maintained on the leg of the subject, one or more subsequent transdermal delivery device may be reapplied to a different position, such as the abdomen or back of the subject. Subsequent dosages applied in multiple dosage interval regimens may have the same or different formulation of naloxone, e.g., with respect to active agent concentration, matrix formulation, etc., as desired.

### Utility

Compositions and methods of the invention, e.g., as described above, find use in any application where transdermal delivery of naloxone to a subject is desired, e.g., to treat a subject for a malady, disease, ailment or condition that can be treated with naloxone. The term “treatment” is used herein in its conventional sense to mean that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely eliminated, such that the subject no longer suffers from the condition, or at least the symptoms that characterize the condition. The term “manage” is used herein in its conventional sense to mean that the symptoms associated with the condition afflicting the subject are at least kept under control (i.e., magnitude of the symptom are kept within a predetermined level), where in some instances the symptoms are ameliorated without eliminating the underlying condition.

In some embodiments, the subject compositions find use in applications where transdermal delivery of naloxone to a subject is used in the prevention of a malady, disease, ailment or condition that is prevented by naloxone. The term “prevention” is used herein in its conventional sense to mean the reduction or complete elimination of the occurrence of a particular condition, such as where the subject transdermal delivery devices are prophylactically applied to the skin surface of a subject and the indicated condition is altogether prevented from occurring or a reduction in the severity of the condition or symptoms associated with the condition is experienced by the subject. Accordingly, prophylactically applying the subject naloxone transdermal delivery device is sufficient to reduce the severity of a condition or symptom associated with the malady, disease, ailment or condition by 5% or more as determined by a qualified health care professional, such as by 10% or more, such as by 15% or more, such as by 25% or more, such as by 50% or more, such as by 75% or more, such as by 90% or more, such as by 95% or more and including by 99% or more. In some embodiments, prophylactically applying the subject naloxone transdermal delivery device is sufficient to altogether eliminate the occurrence or any symptoms associated with the malady, disease, ailment or condition. In some embodiments, prophylactically applying the subject naloxone transdermal delivery device is sufficient to reduce the duration of malady, disease, ailment or condition, such as by 0.1 days or more, such as by 0.5 days or more, such as by 1 day or more, such as by 2 days or more, such as by 3 days or more, such as by 4 days or more, such as by 5 days or more, such as by 6 days or more, such as by 7 days or more and including by 14 days or more.

In some instances, the subject naloxone transdermal delivery devices and methods of using the same are employed in conjunction with opioid therapy to achieve one or more benefits. For example, the subject naloxone transdermal delivery devices and methods of using the same may be employed in conjunction with opioid therapy to treat or prevent one or more opioid side effects, such as but not limited to: opioid induced constipation (OIC), drowsiness, nausea and vomiting, cravings/euphoria, etc. The term “opioid” is used herein in its conventional sense to refer to the naturally occurring or synthetic chemical substances that exert pharmacological action by interaction at opioid receptors (i.e., μ, κ and δ opioid receptors). In certain embodiments opioids are biosynthetic benzylisoquinoline alkaloids and may be opioid receptor agonists, antagonists and inverse agonists.

Constipation may be defined as having one or more symptoms: hard stools, infrequent stools, need for excessive straining, sense of incomplete bowel evacuation, and excessive time spent on the toilet or in unsuccessful defecation. OIC is a type secondary constipation specifically caused by opioids. When employed to treat OIC, application of a naloxone transdermal delivery device to a subject undergoing opioid therapy results in amelioration, if not complete cessation, of one or more OIC symptoms, e.g., as described above. When employed to prevent OIC, application of a naloxone transdermal delivery device to a subject undergoing opioid therapy results in avoidance of one or more OIC symptoms appearing. When used to prevent OIC, a naloxone transdermal delivery device may be administered to a subject undergoing opioid therapy at a time prior to one or more symptoms of OIC appearing, e.g., when the opioid therapy is started.

When used to treat OIC, methods of the invention may include diagnosing a subject as having OIC. Diagnosis of OIC may be performed using any convenient protocol, such as those diagnostic protocols described in Nelson & Camilleri, “Opioid-induced constipation: advances and clinical guidance,” Ther. Adv. Chronic Dis. (2016) 7:121-134. When used to treat OIC, methods of the invention may include assessing a subject for amelioration and/or cessation of one or more OIC symptoms, such as described above.

In some instances, OIC treatment or prevention in accordance with the subject methods is accompanied by negligible, if any, opioid withdrawal symptoms. Opioid withdrawal symptoms include early symptoms, such as muscle aches, restlessness, anxiety, lacrimation (eyes tearing up), runny nose, excessive sweating, inability to sleep, yawning very often, and later symptoms, such as diarrhea, abdominal cramping, goose bumps on the skin, nausea and vomiting, dilated pupils and possibly blurry vision, rapid heartbeat and high blood pressure. In some instances, more severe symptoms of opioid withdrawal are avoided, and mild symptoms may or may not occur.

In some instances, OIC treatment or prevention in accordance with the subject methods is accompanied by negligible, if any, reduction in analgesia. As such, practice of the methods of the invention may result in a reduction in opioid analgesia of 20% or less, e.g., 10% or less, such as 5% or less, including 1% or less.

In some instances, practice of the subject methods in a subject undergoing opioid therapy provides for at least a reduction in the magnitude of opioid dosage increase to achieve adequate pain relief, as compared to a control where an increase in opioid dosage is required to achieve adequate pain relief, e.g., because of tolerance. As such, practice of the subject methods in conjunction with opioid therapy results, in some instances, in the ability to obtain adequate pain relief with a reduced amount of opioid dosage increase, which may no increase in opioid dosage, as well as reduced opioid dosage, as compared to control in which naloxone is not administered to a subject. Practice of the subject methods in combination with opioid therapy may also result in at least a reduction, if not prevention, of the occurrence of opioid addiction.

As such, devices and methods of the invention find use in conjunction with opioid therapy. The opioid active agent of the opioid therapy may vary. Examples of opioid active agents include, but are not limited to: codeine, morphine, oripavine, pseudomorphine, thebaine, 14-dydroxymorphine, 2,4-dinitrophenylmorphine, 6-methyldihydromorphine, 6-methylenedihydrodesoxymorphine, 6-acetyldihydromorphine, azidomorphine, chlornaltrexamine, chloroxymorphamine, desomorphine (dihydrodesoxymorphine), dihydromorphine, ethyldihydromorphine, hydromorphinol, methyldesorphine, N-phenethylnormorphine, RAM-378, 6-nicotinoyldihydromorphine, acetylpropionylmorphine, diacetyldihydromorphine (dihydroheroin, acetylmorphinol), dibutyrylmorphine, dibenzoylmorphine, diformylmorphine, dipropanoylmorphine, heroin (diacetylmorphine), nicomorphine, 6-monoacetylcodeine, benzylmorphine, codeine methylbromide, desocodeine, dimethylmorphine (6-O-methylcodeine), ethyldihydromorphine, methyldihydromorphine (dihydroheterocodeine), ethylmorphine (dionine), heterocodeine, isocodeine, pholcodine (morpholinylethylmorphine), myrophine, nalodeine (N-allyl-norcodeine), transisocodeine, 14-cinnamoyloxycodeinone, 14-ethoxymetopon, 14-methoxymetopon, 14-phenylpropoxymetopon, 7-spiroindanyloxymorphone, 8,14-dihydroxydihydromorphinone, acetylcodone, acetylmorphone, α-hydrocodol, bromoisopropropyldihydromorphinone, codeinone, codorphone, codol, codoxime, IBNtxA, acetyldihydrocodeinone, dihydrocodeinone enol acetate, hydrocodone, hydromorphone, hydroxycodeine, methyldihydromorphinone, morphenol, morphinone, morphol, N-phenethyl-14-ethoxymetopon, oxycodone, oxymorphol, oxymorphone, pentamorphone, semorphone, α-chlorocodide, β-chlorocodide, α-chloromorphide, bromocodide, bromomorphide, chlorodihydrocodide, chloromorphide, codide, 14-hydroxydihydrocodeine, acetyldihydrocodeine, dihydrocodeine, dihydrodesoxycodeine, dihydroisocodeine, nicocodeine, nicodicodeine, 1-nitrocodeine, codeine-N-oxide, morphine-N-oxide, oxymorphazone, 1-bromocodeine, 1-chlorocodeine, 1-iodomorphine, codeine-N-oxide, heroin-7,8-oxide, morphine-6-glucuronide, 6-monoacetylmorphine, morphine-N-oxide, naltrexol, norcodeine, normorphine, 4-chlorophenylpyridomorphinan, cyclorphan, dextrallorphan, levargorphan, levorphanol, levophenacylmorphan, levomethorphan, norlevorphanol, N-methylmorphinan, oxilorphan, phenomorphan, methorphan, morphanol, Ro4-1539, stephodeline, Xorphanol, 1-nitroaknadinine, 14-episinomenine, 5,6-dihydronorsalutaridine, 6-keto nalbuphine, aknadinine, butorphanol, cephakicine, cephasamine, cyprodime, drotebanol, fenfangjine G, nalbuphine, sinococuline, sinomenine, tannagine, 5,9 alpha-diethyl-2-hydroxybenzomorphan (5,9-DEHB), 8-carboxamidocyclazocine (8-CAC), alazocine, anazocine, bremazocine, butinazocine, carbazocine, cogazocine, cyclazocine, dezocine, eptazocine, etazocine, ethylketocyclazocine, fedotozine, fluorophen, gemazocine, ibazocine, ketazocine, metazocine, moxazocine, pentazocine, phenazocine, quadazocine, thiazocine, tonazocine, volazocine, zenazocine, 4-fluoromeperidine, allylnorpethidine, anileridine, benzethidine, carperidine, difenoxin, diphenoxylate, etoxeridine, carbetidine, furethidine, hydroxypethidine, bemidone, morpheridine, meperidine-N-oxide, oxpheneridine, carbamethidine, pethidine, meperidine, norpethidine, pethidinic acid, pheneridine, phenoperidine, piminodine, properidine, ipropethidine, sameridine, allylprodine, (α/β)-meprodine, desmethylprodine (MPPP), PEPAP, (α/β)-prodine, prosidol, trimeperidine (promedol), acetoxyketobemidone, droxypropine, ketobemidone, methylketobemidone, propylketobemidone, alvimopan, loperamide, picenadol, dextromethadone, dipipanone, isomethadone, levoisomethadone, levomethadone, methadone, normethadone, norpipanone, phenadoxone (heptazone), pipidone (6-piperidine-4,4-diphenyl-5-methyl-hexanone-3 hydrochloride), alphaacetylmethadol, dimepheptanol (racemethadol), levacetylmethadol, noracetylmethadol, desmethylmoramide, dextromoramide, levomoramide, moramide intermediate, racemoramide, diethylthiambutene, dimethylthiambutene, ethylmethylthiambutene, piperidylthiambutene, pyrrolidinylthiambutene, thiambutene, tipepidine, dextropropoxyphene (propoxyphene), dimenoxadol, dioxaphetyl butyrate, levopropoxyphene, norpropoxyphene, diampromide, phenampromide, propiram, IC-26, isoaminile, lefetamine, R-4066, 3-allylfentanyl, 3-methylfentanyl, 3-methylthiofentanyl, 4-phenylfentanyl, alfentanil, alphamethylacetylfentanyl, alphamethylfentanyl, alphamethylthiofentanyl, benzylfentanyl, betahydroxyfentanyl, betahydroxythiofentanyl, betamethylfentanyl, brifentanil, carfentanil, fentanyl, lofentanil, mirfentanil, ocfentanil, ohmefentanyl, parafluorofentanyl, phenaridine, remifentanil, sufentanil, thenylfentanyl, thiofentanyl, trefentanil, 7-PET, acetorphine, alletorphine (N-allyl-noretorphine), BU-48, dexmedetomidine, cyprenorphine, dihydroetorphine, etorphine, homprenorphine, 18,19-dehydrodexmedetomidine, N-cyclopropylmethylnoretorphine, nepenthone, nordexmedetomidine, thevinone, thienorphine, ethoheptazine, meptazinol, metheptazine, metethoheptazine, proheptazine, bezitramide, piritramide, clonitazene, etonitazene, nitazene, 18-methoxycoronaridine, 7-acetoxymitragynine, 7-hydroxymitragynine, akuammidine, akuammine, eseroline, hodgkinsine, mitragynine, pericine, pseudoakuammigine, BW373U86, DPI-221, DPI-287, DPI-3290, SNC-80, dynorphin A, dynorphin B, β-endorphin, α-endorphin, γ-endorphin.α-neo-endorphin.β-neo-endorphin, DADLE, DAMGO, dermenkephalin, met-enkephalin, leu-enkephalin, adrenorphin, amidorphin, casomorphin, DALDA (Tyr-D-Arg-Phe-Lys-NH2), deltorphin, dermorphin, DPDPE, endomorphin, gliadorphin, morphiceptin, nociception, octreotide, opiorphin, rubiscolin, TRIMU 5, 3-(3-methoxyphenyl)-3-ethoxycarbonyltropane, AD-1211, AH-7921, azaprocin, BDPC, bisnortilidine, BRL-52537, bromadoline, C-8813, ciramadol, doxpicomine, enadoline, faxeladol, GR-89696, herkinorin, IC-199,441, ICI-204,448, J-113,397, JTC-801, ketamine, KNT-42, LPK-26, methopholine, MT-45, desmethylclozapine, NNC 63-0532, nortilidine, O-desmethyltramadol, phenadone, phencyclidine, prodilidine, profadol, Ro64-6198, salvinorin A, SB-612,111, SC-17599, RWJ-394,674, TAN-67, tapentadol, oxycodone, tifluadom, tilidine, tramadol, trimebutine, U-50,488, U-69,593, viminol, 1-(4-nitrophenylethyl)piperidylidene-2-(4-chlorophenyl)sulfonamide (W-18), 5′-guanidinonaltrindole, β-funaltrexamine, 6β-naltrexol, alvimopan, binaltorphimine, chlornaltrexamine, clocinnamox, cyclazocine, cyprodime, diacetylnalorphine, difenamizole, diprenorphine, fedotozine, JDTic, levallorphan, methocinnamox, methylnaltrexone, nalfurafine, nalmefene, nalmexone, naloxazone, naloxonazine, naloxone, naloxone benzoylhydrazone, nalorphine, naltrexone, naltriben, naltrindole, norbinaltorphimine, oxilorphan, S-allyl-3-hydroxy-17-thioniamorphinan, alimadol, anilopam HCl, asimadoline, FE 200665, fedotozine, MCOPPB, nalfurafine, nalorphine, nalorphine dinicotinate, SoRI-9409, among other opioids.

The terms “in conjunction with,” “co-administration” and “in combination with” include the administration of a naloxone transdermal delivery device of the invention and an opioid either simultaneously, concurrently or sequentially within no specific time limits. In certain embodiments, a naloxone transdermal delivery device can be administered prior to (e.g., minutes, 15 minutes, 30 minutes, 45 minutes, 1 hour, 2 hours, 4 hours, 6 hours, 12 hours, 24 hours, 48 hours, 72 hours, or 96 hours before), concomitantly with, or subsequent to (e.g., 5 minutes, minutes, 30 minutes, 45 minutes, 1 hour, 2 hours, 4 hours, 6 hours, 12 hours, 24 hours, 48 hours, 72 hours, 96 hours, or 1 week or longer after) the administration of an opioid. “Concomitant administration” of an opioid with a naloxone transdermal delivery device of the invention means administration of the opioid and the naloxone transdermal delivery device at such time that both the opioid and naloxone will have a therapeutic effect. Such concomitant administration may involve concurrent (i.e. at the same time), prior, or subsequent administration of the opioid with respect to the administration of the naloxone transdermal delivery device. In some embodiments, the naloxone transdermal delivery device and the opioid are administered to the subject within twenty-four hours of each other, such as within 12 hours of each other, within 6 hours of each other, within 3 hours of each other, or within 1 hour of each other. In certain embodiments, the naloxone transdermal delivery device and the opioid are administered within 1 hour of each other. In certain embodiments, the naloxone transdermal delivery device and the opioid are administered substantially simultaneously. By administered substantially simultaneously is meant that the naloxone transdermal delivery device and the opioid are administered to the subject within about 10 minutes or less of each other, such as 5 minutes or less, or 1 minute or less of each other.

In certain embodiments, the applied naloxone transdermal delivery device as described herein when co-administered with an opioid may reduce the amount of opioid required to effectively treat or manage pain. In some instances, applying the naloxone transdermal delivery device according to the methods described above reduces the amount of co-administered opioid needed to manage pain by 1% or more by weight, such as by 2% or more by weight, such as by 3% or more by weight, such as by 5% or more by weight, such as by 10% or more by weight, such as by 15% or more by weight, such as by 25% or more by weight and including reducing the amount of co-administered opioid needed to manage pain by 50% or more by weight. In other words, the amount of opioid needed to manage the pain is reduced by 1% or more by weight as compared to the amount of opioid alone that is needed to manage pain, such as by 2% or more by weight, such as by 3% or more by weight, such as by 5% or more by weight, such as by 10% or more by weight, such as by 15% or more by weight, such as by 25% or more by weight and including where the amount of opioid needed to manage pain is reduced by 50% or more by weight as compared to the amount of opioid alone that is needed to manage the pain.

In certain embodiments, methods include replacing one or more dosages of administered opioids (e.g., opioid agonists) in an opioid pain-management regimen with a transdermal delivery device containing naloxone as described herein. The term “opioid pain-management regimen” refers to pain-management protocols (e.g., by a healthcare professional in a medical care facility, under the supervision by a health care professional or at a subject's home under the direction/prescription of a health care professional) having scheduled dose administrations of opioids to manage pain in the subject. For example, opioid pain-management regimens of interest may include pain-management protocols which employ a plurality of scheduled administrations of opioid dosages to manage, reduce or eliminate acute pain, chronic pain, neuropathic pain, pain associated with cancer, postoperative pain, moderate to severe pain, labor pain, perioperative pain, among other types of pain known for being managed my opioids. In certain embodiments, the opioid pain-management regimen replaced or supplemented by applying a transdermal delivery device containing naloxone as described herein is moderate-to-severe pain that typically require opioids and for which alternative pain alleviation protocols have proven inadequate.

Methods according to these embodiments include replacing one or more scheduled administration of opioids by applying a transdermal delivery device containing naloxone as described herein, such as 2 or more, such as 3 or more, such as 4 or more, such as 5 or more, such as 6 or more, such as 7 or more, such as 8 or more, such as 9 or more, such as 10 or more, such as 15 or more, such as 25 or more, such as 50 or more, such as 75 or more and including 100 or more scheduled administrations of opioids to manage pain. As such, applying and maintaining the transdermal delivery device containing naloxone as described herein may be sufficient to reduce the number of scheduled administrations of opioids in the opioid pain-management regimen by 5% or more, such as by 10% or more, such as by 15% or more, such as by 20% or more, such as by 25% or more, such as by 50% or more, such as by 75% or more, such as by 90% or more, such as by 95% or more and including reducing the number of scheduled administrations of opioids in the opioid pain-management regimen by 99% or more.

In certain embodiments, the subject methods are suitable for entirely replacing opioids (i.e., 100% of the administered opioid dosages) in the opioid pain-management regimen with a transdermal delivery device containing naloxone as described herein. In certain embodiments, methods include applying and maintaining one or more of the subject transdermal delivery devices containing naloxone in conjunction with administration of opioids in the opioid pain-management regimen, as described above. In some instances, a reduced dosage of opioid may be administered to the subject in the opioid pain-management regimen when the naloxone transdermal delivery device is in contact with the subject, such as where the dosage of each schedule administration of opioid in the opioid pain-management regimen is reduced by 5% or more, such as by 10% or more, such as by 15% or more, such as by 20% or more, such as by 25% or more, such as by 50% or more and including by 75% or more. In some instances, one or more of the scheduled doses of opioid administration is eliminated (i.e., skipped), such as every other scheduled dose of opioid administration, every 2 scheduled doses, every 3 scheduled doses, every 4 scheduled doses or some other interval. In certain instances, applying one of or more of the subject naloxone transdermal delivery devices is sufficient to eliminate sequential scheduled doses of opioid administration in the opioid pain-management regimen, such as 2 or more sequentially scheduled doses, such as 3 or more sequentially scheduled doses and include 4 or more sequentially scheduled doses of opioid administration in the opioid pain-management regimen.

In some instances, the subject naloxone transdermal delivery devices and methods described herein are employed in the treatment of atopic dermatitis. In some embodiments, the atopic dermatitis is treated systemically with one or more of the naloxone transdermal delivery devices, such as where the transdermal delivery device is applied to the skin of a subject at a location different from the atopic dermatitis. For example, the transdermal delivery device may be positioned on the arm or leg of the subject where the atopic dermatitis is positioned on another part of the body, such as the head or torso. In these embodiments where the atopic dermatitis is treated with systemic naloxone, the transdermal delivery device may be positioned at a location that is 1 cm or more away from the atopic dermatitis, such as 2 cm or more, such as 5 cm or more, such as 10 cm or more, such as 15 cm or more, such as 25 cm or more, such as 50 cm or more and including 100 cm or more away.

In other embodiments, the subject transdermal delivery devices may be applied to the area affected by the atopic dermatitis to deliver a local therapeutic effect. For example, the transdermal delivery device may cover 5% or more of the affected area of the skin, such as 10% or more, such as 15% or more, such as 25% or more, such as 50% or more, such as 75% or more and including 90% or more of the affected area of the skin. In certain embodiments, the transdermal delivery device covers the entire area affected by atopic dermatitis to deliver a local therapeutic effect. Where a transdermal naloxone patch is used to locally treat atopic dermatitis, the adhesive maybe mechanically gentle to the skin such as where the excipients result in little-to-no skin irritation.

In other instances, the subject naloxone transdermal delivery devices and methods described herein are employed in the treatment of pruritus. The term pruritus is used herein in its conventional sense to refer to the unpleasant sensation that provokes the desire to scratch and may include, but is not limited to, renal pruritus, cholestatic pruritus, hematologic pruritus, endocrine pruritus, pruritus related to malignancy, idiopathic generalized pruritus or a combination thereof. The pruritus may, in certain embodiments, be associated with a primary skin disorder, such as xerosis, atopic dermatitis, urticaria, psoriasis, arthropod assault, mastocytosis, dermatitis herpetiformis, or pemphigoid. The subject naloxone transdermal delivery devices and methods, in some embodiments, reduce the duration of pruritus experienced by the subject, such as by 5% or more, such as by 10% or more, such as by 25% or more, such as by 50% or more, such as by 75% or more, such as by 90% or more and including by 99% or more. In other embodiments, the subject naloxone transdermal delivery devices and methods reduce the severity of the pruritus experienced by the subject, such as by 5% or more, such as by 10% or more, such as by 25% or more, such as by 50% or more, such as by 75% or more, such as by 90% or more and including by 99% or more. In certain embodiments, the subject naloxone transdermal delivery devices and methods entirely eliminates the pruritus experienced by the subject.

The present disclosure also provides for transdermal delivery devices having a naloxone composition for use in the treatment of a subject. In some embodiments, the present disclosure provides for transdermal delivery devices having a naloxone composition for use in the treatment or prevention of opioid side effects, such as but not limited to: opioid induced constipation (OIC), drowsiness, nausea and vomiting, cravings/euphoria, etc. In other embodiments, the present disclosure provides for transdermal delivery devices having a naloxone composition for use in the treatment of atopic dermatitis. In still other embodiments, the present disclosure provides for transdermal delivery devices having a naloxone composition for use in the treatment of pruritus, such as renal pruritus, cholestatic pruritus, hematologic pruritus, endocrine pruritus, pruritus related to malignancy, idiopathic generalized pruritus or a combination thereof, as well as pruritus be associated with a primary skin disorder, such as xerosis, atopic dermatitis, urticaria, psoriasis, arthropod assault, mastocytosis, dermatitis herpetiformis, or pemphigoid.

Compositions and methods of the invention, e.g., as described above, also provide for the use of the subject naloxone compositions for the manufacture of a transdermal delivery device. In some embodiments, the present disclosure provides for the use of the subject naloxone compositions for the manufacture of a transdermal delivery device for the treatment or prevention of opioid side effects, such as but not limited to: opioid induced constipation (OIC), drowsiness, nausea and vomiting, cravings/euphoria, etc. In other embodiments, the present disclosure provides for the use of the subject naloxone compositions for the manufacture of a transdermal delivery device for the treatment of atopic dermatitis. In yet other embodiments, the present disclosure provides the use of the subject naloxone compositions for the manufacture of a transdermal delivery device for the treatment of pruritis. In these embodiments, the pruritus may be renal pruritus, cholestatic pruritus, hematologic pruritus, endocrine pruritus, pruritus related to malignancy, idiopathic generalized pruritus or a combination thereof, as well as pruritus be associated with a primary skin disorder, such as xerosis, atopic dermatitis, urticaria, psoriasis, arthropod assault, mastocytosis, dermatitis herpetiformis, or pemphigoid.

### Kits

Kits for use in practicing certain methods described herein are also provided. In certain aspects, the kits include one or more of any of the transdermal compositions described elsewhere herein. According to certain aspects, the kits include two or more of the subject transdermal compositions. In a given kit that includes two or more compositions, the compositions may be individually packaged or present within a common container, where the packaging and/or container may be sterile, e.g., a sterile pouch.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### I. Naloxone Transdermal Formulations

### A. Preparation of Formulations

Formulations were prepared by mixing stock solutions of each of the mixture components in organic solvents (typically 30-60 wt % solid content in ethyl acetate, methanol and/or ethanol), followed by a mixing process. Once a homogeneous mixture was formed, the solution was cast on a release liner (siliconized polyester sheet of 2-3 mils) and dried at 650-80° C. for 10-90 minutes. The adhesive films were then laminated to a PET backing, cut to the desired size, and pouched.

The following formulations were prepared and tested:

| No. | Formulation |
|---|---|
| 0 | Clonidine |
| 1 | 8% Naloxone/10% PVP/5% LauricAcid |
| | DT-9301 |
| 2 | 8% Naloxone/10% PVP/5% LaurylAlcohol |
| | DT-9301 |
| 3 | 8% Naloxone/10% PVP/3% LauricAcid/3% LaurylAlcohol |
| | DT-9301 |
| 4 | 12% Naloxone/10% PVP/5% LauricAcid/5% PropyleneGlycol |
| | DT-9301 |
| 5 | 12% Naloxone/10% PV/5% LaurylAlcohol/2.5% PropyleneGlycol |
| | DT-9301 |
| 6 | 5% Naloxone/10% PVP/5% Lauric Acid/2.5% PropyleneGlycol |
| | DT-9301 |
| 7 | 5% Naloxone/10% PVP/5% LaurylAlcohol/2.5% PropyleneGlycol |
| | DT-9301 |
| 8 | 8% Naloxone/10% PVP K90 |
| | DT-9301 |

### B. Transdermal Flux Tests

Human cadaver skin was used and the isolated epidermal layers (stratum corneum and viable epidermis) were heat separated from the full-thickness skin as skin membrane. Samples were die-cut with an arch punch to a final diameter of about 2.0 cm². The release liner was removed and the system was placed on top of the epidermis/stratum corneum with the drug adhesive layer facing the stratum corneum. Gentle pressure was applied to effect good contact between the adhesive layer and stratum corneum. The donor and receptor sides of the Franz cell were clamped together and the receptor solution containing a phosphate buffer at pH 6.5 was added to the Franz cell. The cells were kept at 33° C. for the duration of the experiment. Samples of the receptor solution were taken at regular intervals and the active agent concentration was measured by HPLC. The removed receptor solution was replaced with fresh solution to maintain the sink conditions. The average flux was calculated from the slope of cumulative amounts of the drug in the receiver compartment versus time plot.

### C. Results

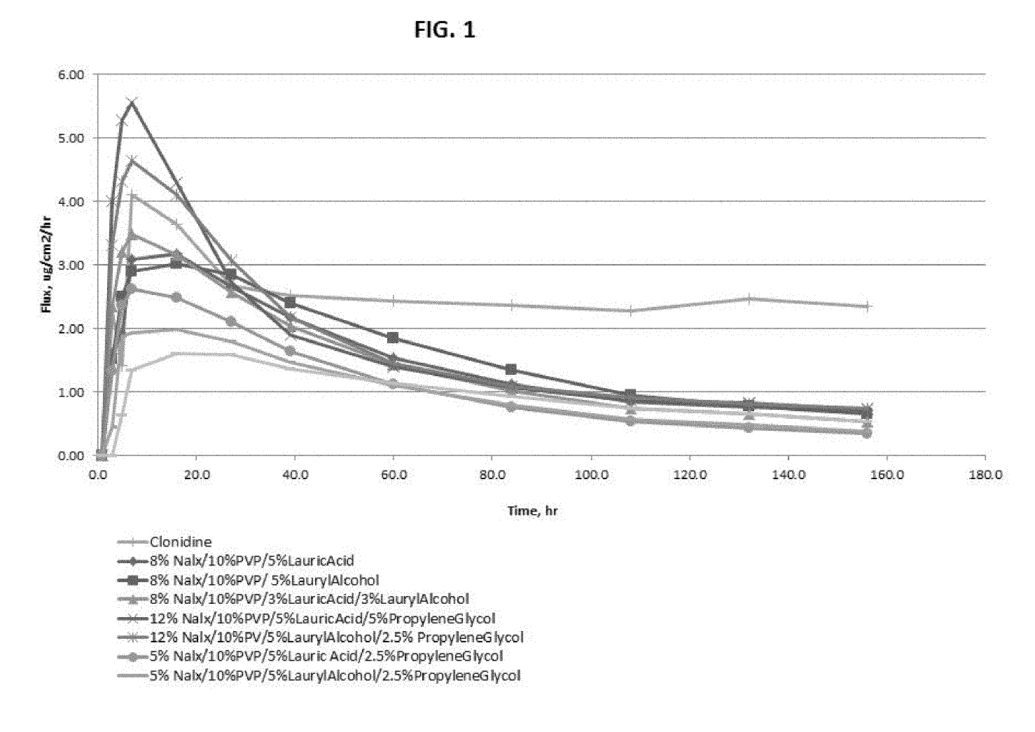

FIG. 1 provides the mean flux for a given formulation calculated from the slopes of the cumulative amounts of the drug in the receiver compartment versus time plot for each diffusion cell for that tested formulations.

### II. Evaluation of Dermal Irritation and Pharmacokinetics of Two Transdermal Naloxone Formulations in Naïve Female Gottingen Mini Pigs

### A. Introduction

The purpose of this study was to determine the dermal irritation and pharmacokinetics of two transdermal naloxone formulations when worn for three days by naïve female Gottingen mini-pigs and to compare the pharmacokinetic transdermal profiles to the PK of intravenously (IV) administered naloxone.

In this study, the dermal irritation (Primary Irritation Index or PII) of these two transdermal naloxone formulations was investigated. Additionally, the pharmacokinetics of the transdermal naloxone formulations were determined and compared to the pharmacokinetic profiles of intravenously (IV) administered naloxone. Transdermal Formulation 1 (8% Naloxone/10% PVP K90/4% Lauric Acid/78% DuroTak 9301) resulted in no observed skin irritation (PII score 0) compared to Formulation 2 (6% Naloxone/10% PVP K90/6% Myristic Acid/78% DuroTak 9301 which resulted in negligible observed skin irritation (PII score 0.3). Pharmacokinetic analyses showed generally similar exposures between Formulation 1 (Lot 177-65-1) and Formulation 2 (Lot 177-65-2) for all AUC parameters calculated. Similarly, Cmax and Tmax were also approximately equal between the two formulations. Based on the results of this study, there were minor and insignificant differences between patch formulations of Naloxone for erythema, edema, and general observations of the affected skin and showed similar exposure. In general, both formulations were well-tolerated by the animals for the duration of the study.

### B. Materials and Methods

### 1. Formulations

a. Naloxone (Transdermal Patch Formulation 1): 8% Naloxone/10% PVP K90/4% Lauric Acid/78% DuroTak 9301
b. Naloxone (Transdermal Patch Formulation 2): 6% Naloxone/10% PVP K90/6% Myristic Acid/78% DuroTak 9301
c. Naloxone (IV formulation) Naloxone 0.4 mg/ml (Hospira)(Lot 50-283-EV)

### 2. Dose Preparation and Concentration

Test materials (Naloxone Transdermal Formulations 1 & 2) were in the form of a single patch that was applied to the prepared skin site of the animals and remained on for a period of three (3) days.

The positive control article (marketed formulation), Naloxone (Narcan 0.4 mg/mL) was diluted with sterile 0.9% normal saline to a concentration of 0.04 mg/mL in order to achieve a desired dose volume of 0.25 mL/kg and dosage of 0.01 mg/kg.

### 3. Experimental Design

Gottingen mini-pigs were employed. The live-phase portion of this study was seven (7) days in duration.

The day before transdermal patch administration, the hair was carefully clipped close to the skin from the dorsal trunk area where the patch was applied.

The site was washed with chlorhexidine followed by warm water and blotted dry.

On the day of dosing, the patch sites were prepared by washing the area with warmed chlorhexidine scrub to remove all detritus, followed by wiping the areas with warmed sponges soaked in warm water, gently blotting with dry gauze pads, and allowing to air-dry before application. The patch was applied to an area free of abrasions.

Groups 1 and 2: A single transdermal patch containing one of two distinct Naloxone formulations was placed on the dorsal trunk of the mini-pig (right side), secured in place, overlaid with Tegaderm and further secured with Vetwrap and Elasticon tape. A jacket was also used to maintain the patches in place. Each transdermal patch was maintained in place for three days.

Group 3: Narcan intravenous formulation (0.4 mg/mL) was diluted with sterile 0.9% normal saline to a concentration of 0.04 mg/mL. The dose volumes were calculated using the individual body weights of the pigs on the day of dosing. Narcan was administered as an IV bolus dose via the marginal ear vein at 0.01 mg/kg and 0.25 mL/kg (BTS SOP E50.01). Group 3 served as a positive control group.

The administration of test compounds is summarized in Table 1, below.

TABLE 1

| Group | | Test | Dose | | Dose | Dosing |
|---|---|---|---|---|---|---|
| No. | N | Compound | Route | Dose | Volume | Schedule |
| 1 | 3 | Transdermal | Topical | 3 | NA | Single |
| | | Formulation 1: | (30 cm² | mg/day | | admin for |
| | | 8% Naloxone | patch) | | | 3 days |
| | | 10% PVP K90 | | | | |
| | | 4% Lauric | | | | |
| | | Acid | | | | |
| | | 78% DuroTak | | | | |
| | | 9301 | | | | |
| 2 | 3 | Transdermal | Topical | 3 | NA | Single |
| | | Formulation 2: | (30 cm² | mg/day | | admin for |
| | | 6% Naloxone | patch) | | | 3 days |
| | | 10% PVP K90 | | | | |
| | | 6% Myristic | | | | |
| | | Acid | | | | |
| | | 78% DuroTak | | | | |
| | | 9301 | | | | |
| 3 | 3 | Narcan (IV | Intra- | 0.01 | 0.25 | Single |
| | | formulation) | venous | mg/kg | ml/kg | admin |

Animals were monitored at least twice a day while wearing the patch to ensure that the patch remained in place. No patches fell off during the course of the study.

Animals were lightly anesthetized using Isoflurane as an inhalation anesthetic (BTS SOP E54) for the purpose of Test Material Patch application, intravenous administration, and for test site scoring.

The time of Test Material patch application and removal were recorded for each animal so that dermal scoring and PK time-points were calculated. The time of intravenous injection was also recorded so that PK time-points were calculated.

### C. Results

### 1. Clinical Observations and Evaluation of Sites

In general, the formulations used on this study were well-tolerated by the animals. On Days 5, 6, and 7, one test animal was observed to excrete unformed feces green in color. All other animals on study were observed to be normal clinically. All animals were observed to have tan to slight tan skin discoloration on the application site post-removal and which generally resolved 24 hour post patch removal. This accumulation of brown exudate is commonly found in Gottingen pigs with occlusive patches. Two animals from Group 2 had slight erythema scores (score 1). The PII for Group 1 was 0 (none) and the PII for Group 2 was 0.3 (negligible) as summarized below in Table 2.

TABLE 2

| | | Erythema/ | Erythema/ | |
|---|---|---|---|---|
| | | Edema | Edema | Primary |
| | Animal | 1 Hour | 48 Hour | Irritation |
| | Number | PPR** | PPR** | Index** |
| Group 1 | | | | |
| Formulation 1: | | | | |
| 8% Naloxone | | | | |
| 10% PVP K90 | 1F001 | 0 | 0 | 0 |
| 4% Lauric Acid | 1F002 | 0 | 0 | |
| 78% DuroTak 9301 | 1F003 | 0 | 0 | |
| (Lot No. 177-65-1) | Average | 0.0 | 0.0 | |
| | Per Animal | | | |
| Group 2 | | | | |
| Formulation 2: | | | | |
| 6% Naloxone | | | | |
| 10% PVP K90 | 2F001 | 0 | 1 | 0 |
| 6% Myristic Acid | 2F002 | 0 | 0 | |
| 78% DuroTak 9301 | 2F003 | 0 | 1 | |
| (Lot No. 177-65-02) | Average | 0.0 | 0.7 | |
| | Per Animal | | | |

### 2. Pharmacokinetics

a. Transdermal Patch Formulations (Groups 1 and 2)

Exposures of animals to naloxone were similar following treatment with either Formulation 1 (Lot 177-65-1) or Formulation 2 (Lot 177-65-2). The Cmax was 227±149 and 253±142 μg/mL for Formulations 1 and 2, respectively. The Tmax (hours) was 28±18 and 32±14 for Formulations 1 and 2, respectively. All AUC parameters were also similar. The AUC(0-24) was 3232 and 3104 μg/mL*hr and the AUC(0-144) was 14488 and 12876 μg/mL*hr for Formulations 1 and 2, respectively.

Similarly, comparison of the AUC(0-24) between transdermally administered naloxone and intravenously administered naloxone was similar between Formulations 1 and 2. The ratio was 3.39±3.29 for Formulation 1 and 3.26±1.73 for Formulation 2.

b. Intravenously Administered Naloxone (Group 3)

Exposure following an intravenous bolus of naloxone was typical of IV administered drugs. The Tmax and Cmax occurred at the first time point collected. The Tmax was 5 minutes post dose.

### D. Discussion and Conclusion

Body weight change was unremarkable for all animals. There was incidence of diarrhea in one animal. No other clinical signs were present in the remaining groups. After removal of the test patches, tan to slight tan discoloration on application site was observed which is common for Gottingen minipigs with occlusive patches.

Naloxone patches applied topically for three days for Formulations 1 (8% Naloxone/10% PVP K90/4% Lauric Acid/78% DuroTak 9301) & 2 (6% Naloxone/10% PVP K90/6% Myristic Acid/78% DuroTak 9301) both are classified as none—negligible irritants with a PII=0 and PII=0.3 respectively.

CBC parameters and clinical chemistry measures were largely within normal ranges in all groups during acclimation, pre-dose Day −1, and 24 hr post patch removal (Group 1 & 2) or 24 hr post injection (Group 3).

Pharmacokinetic analysis showed generally similar exposure between Formulation 1 (Lot 177-65-1) and Formulation 2 (Lot 177-65-2) for all the AUC parameters calculated. Similarly, Cmax and Tmax were also approximately equal between the two formulations. Based on the results of this study, there were minor differences between patch formulations of Naloxone for erythema, edema, and general observations of the affected skin and showed similar exposure. Treatment was generally well-tolerated over the course of this study.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A transdermal delivery device comprising:
a matrix comprising a naloxone active agent in a pressure sensitive adhesive; and
a backing layer.

2. The transdermal delivery device according to claim 1, wherein the device is configured to deliver the naloxone active agent to a subject over an extended period of time.

3. The transdermal delivery device according to claim 2, wherein the extended period of time is 1 to 7 days.

4-5. (canceled)

6. The transdermal delivery device according to claim 1, wherein the amount of naloxone active agent in the matrix adhesive layer is 20% w/w or less.

7. (canceled)

8. The transdermal delivery device according to claim 1, wherein the naloxone active agent is naloxone free base.

9. The transdermal delivery device according to claim 1, wherein the device is configured to deliver an amount of the naloxone active agent to a subject at a rate ranging from 0.1 μg/day to 10,000 μg/day.

10. (canceled)

11. The transdermal delivery device according to claim 1, wherein the pressure sensitive adhesive comprises an acrylic polymer, acrylate copolymer, acrylate-vinyl acetate copolymer or polyacrylonitrile or mixtures thereof.

12. The transdermal delivery device according to claim 11, wherein the pressure sensitive adhesive comprises a non-functionalized polymer.

13. The transdermal delivery device according to claim 12, wherein the pressure sensitive adhesive comprises a non-functionalized acrylate polymer.

14. The transdermal delivery device according to claim 1, wherein the matrix comprises a cross-linked polymer.

15. The transdermal delivery device according to claim 14, wherein the cross-linked polymer is a crosslinked polyvinylpyrrolidone.

16. (canceled)

17. The transdermal delivery device according to claim 1, wherein the matrix comprises a skin penetration enhancer.

18. The transdermal delivery device according to claim 17, wherein the skin penetration enhancer comprises a fatty acid.

19. The transdermal delivery device according to claim 18, wherein the fatty acid is a saturated fatty acid.

20. The transdermal delivery device according to claim 19, wherein the saturated fatty acid is a C8 to C16 fatty acid.

21-22. (canceled)

23. A method administering naloxone to a subject, the method comprising:
applying to a skin surface of the subject a transdermal delivery device comprising:
a matrix comprising a naloxone active agent in a pressure sensitive adhesive; and
a backing layer;
to administer naloxone to the subject.

24-59. (canceled)

60. The method according to claim 23, wherein the method is a method for the treatment or prevention of a side effect from opioid administration in a subject.

61. (canceled)

62. The method according to claim 23, wherein the method is a method for the treatment of opioid addiction in a subject.

63. The method according to claim 23, wherein the method is a method for the treatment of a subject for atopic dermatitis.

64. The method according to claim 23, wherein the method is a method for the treatment or prevention of pruritus in a subject.

65. (canceled)
